(19) United States
(12) Patent Application Publication
(10) Pub. No.: US 2007/0015817 A1
(43) Pub. Date: Jan. 18, 2007

(54) 4-((4-(2-pyrrolidinylethoxy)phenyl)methyl)-3-(4-(trifluoromethyl)phenyl)-7-hydroxychromen-2one, pharmaceutically acceptable salts thereof and methods of use therewith

(76) Inventors: Jeffrey A. McKie, San Diego, CA (US); Shripad S. Bhagwat, San Diego, CA (US)

Correspondence Address: JONES DAY, 222 EAST 41ST ST, NEW YORK, NY, 10017, US

(21) Appl. No.: 11/523,373

(22) Filed: Sep. 18, 2006

Related U.S. Application Data
(63) Continuation of application No. 10/412,997, filed on Apr. 14, 2003.
Application No. 10/412,997 is a continuation-in-part of application No. 10/125,965, filed on Apr. 19, 2002, now Pat. No. 6,620,838.

(51) Int. Cl. A61K 31/4025; C07D 405/02

(52) U.S. Cl. 514/422; 548/525

(57) ABSTRACT

Benzopyranone compounds having the following structure: 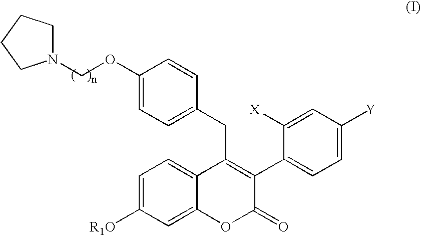
wherein R₁, X, Y and n are as defined here, are disclosed. The compounds of formula (I), wherein R₁ is H, can be prepared by demethylation of the corresponding phenolic methyl ether. The compounds are useful for treating a bone-resorbing disease, cancer, arthritis or an estrogen-related condition such as breast cancer, osteoporosis, endometriosis, cardiovascular disease, hypercholesterolemia, prostatic hypertrophy, prostatic carcinomas, obesity, hot flashes, skin effects, mood swings, memory loss, and adverse reproductive effects associated with exposure to environmental chemicals or natural hormonal imbalances.

This application is a continuation of U.S. application Ser. No. 10/412,997, filed Apr. 14, 2003 which is a continuation-in-part of U.S. application Ser. No. 10/125,965 filed Apr. 19, 2002, now U.S. Pat. No. 6,620,838, issued Sep. 16, 2003 which is incorporated by reference herein in its entirety.

### 1. FIELD OF THE INVENTION

This invention is generally directed to benzopyranone compounds, compositions comprising the benzopyranone compounds and methods for treating a bone-resorbing disease, cancer, arthritis or an estrogen-related condition, comprising administering an effective amount of a benzopyranone compound to a patient in need thereof.

### 2. BACKGROUND OF THE INVENTION

The estrogen hormone has a broad spectrum of effects on tissues in both females and males. Many of these biological effects are positive, including maintenance of bone density, cardiovascular protection, central nervous system (CNS) function, and the protection of organ systems from the effects of aging. However, in addition to its positive effects, estrogen also is a potent growth factor in the breast and endometrium that increases the risk of cancer.

Until recently, it was assumed that estrogen binds to a single estrogen receptor (ER) in cells. As discussed below, this simple view changed significantly when a second ER (ER-β) was cloned (with the original ER being renamed ER-α), and when co-factors that modulate the ER response were discovered. Ligands can bind to two different ERs which, in the presence of tissue-specific co-activators and/or co-repressors, bind to an estrogen response element in the regulatory region of genes or to other transcription factors. Given the complexity of ER signaling, along with the tissue-specific expression of ER-α and ER-β and its co-factors, it is now recognized that ER ligands can act as estrogen agonists and antagonists that mimic the positive effects, or block the negative effects, of estrogen in a tissue-specific manner. This has given rise to the discovery of an entirely new class of drugs, referred to as Selective Estrogen Receptor Modulators or SERMs. These drugs have significant potential for the prevention and/or treatment of cancer and osteoporosis, as well as cardiovascular diseases and neurodegenerative diseases such as Alzheimer's disease.

Bone-resorbing diseases, such as osteoporosis, are debilitating conditions which affect a wide population, and to which there is only limited treatment. For example, osteoporosis affects about 50% of women, and about 10% of men, over the age of 50 in the United States. In individuals with osteoporosis, increased loss of bone mass results in fragile bones and, as a result, increased risk of bone fractures. Other bone-resorption diseases, such as Paget's disease and metastatic bone cancer, present similar symptoms.

Bone is a living tissue which contains several different types of cells. In healthy individuals, the amount of bone made by the osteoblastic cells is balanced by the amount of bone removed or resorbed by the osteoclastic cells. In individuals suffering from a bone-resorbing disease, there is an imbalance in the function of these two types of cells. Perhaps the most well known example of such an imbalance is the rapid increase in bone resorption experienced by postmenopausal women. Such accelerated bone lose is attributed to estrogen deficiency associated with menopause. However, the mechanism of how the loss of estrogen results in increased bone resorption has long been debated.

Recently, investigators have suggested that an increase in bone-resorbing cytokines, such as interleukin-1 (IL-1) and tumor necrosis factor (TNF), may be responsible for postmenopausal bone loss (Kimble et al., J. Biol. Chem. 271:28890-28897, 1996), and that inhibitors of these cytokines can partially diminish bone loss following ovariectomy in rodents (Pacifici, J. Bone Miner Res. 11:1043-1051, 1996). Further, discontinuation of estrogen has been reported to lead to an increase in IL-6 secretion by murine bone marrow and bone cells (Girasole et al., J. Clin. Invest. 89:883-891, 1992; Jilka et al., Science 257:88-91, 1992; Kimble et al., Endocrinology 136:3054-3061, 1995; Passseri et al., Endocrinology 133:822-828, 1993), antibodies against IL-6 can inhibit the increase in osteoclast precursors occurring in estrogen-depleted mice (Girasole et al, supra), and bone loss following ovariectomy does not occur in transgenic mice lacking IL-6 (Poli et al., EMBO J. 13:1189-1196, 1994).

Existing treatments for slowing bone loss generally involves administration of compounds such as estrogen, bisphosphonates, calcitonin, and raloxifene. These compounds, however, are generally used for long-term treatments, and have undesirable side effects. Further, such treatments are typically directed to the activity of mature osteoclasts, rather than reducing their formation. For example, estrogen induces the apoptosis of osteoclasts, while calcitonin causes the osteoclasts to shrink and detach from the surface of the bone (Hughes et al., Nat. Med. 2:1132-1136, 1996; Jilka et al., Exp. Hematol. 23:500-506, 1995). Similarly, bisphosphonates decrease osteoclast activity, change their morphology, and increase the apoptosis of osteoclasts (Parfitt et al., J. Bone Miner Res. 11: 150-159, 1996; Suzuki et al., Endocrinology 137:4685-4690, 1996).

Cytokines are also believed to play an important role in a variety of cancers. For example, in the context of prostate cancer, researchers have shown IL-6 to be an autocrine/paracrine growth factor (Seigall et al., Cancer Res. 50:7786, 1999), to enhance survival of tumors (Okamoto et al., Cancer Res. 57:141-146, 1997), and that neutralizing IL-6 antibodies reduce cell proliferation (Okamoto et al., Endocrinology 138:5071-5073, 1997; Borsellino et al., Proc. Annu. Meet. Am. Assoc. Cancer Res. 37:A2801, 1996). Similar results have been reported for IL-6 with regard to multiple myeloma (Martinez-Maza et al., Res. Immunol. 143:764-769, 1992; Kawano et al., Blood 73:517-526, 1989; Zhang et al., Blood 74:11-13, 1989; Garrett et al., Bone 20:515-520, 1997; and Klein et al., Blood 78:1198-12-4, 1991), renal cell carcinoma (Koo et al., Cancer Immunol. 35:97-105, 1992; Tsukamoto et al., J Urol. 148:1778-1782, 1992; and Weissglas et al., Endocrinology 138:1879-1885, 1997), and cervical carcinoma (Estuce et al., Gynecol. Oncol. 50:15-19, 1993; Tartour et al., Cancer Res. 54:6243-6248, 1994; and Iglesias et al., Am. J. Pathology 146:944-952, 1995).

Furthermore, IL-6 is also believed to be involved in arthritis, particularly in adjuvant-, collagen- and antigen-induced arthritis (Alonzi et al., J. Exp. Med. 187:146-148, 1998; Ohshima et al., Proc. Natl. Acad. Sci. USA 95:8222-8226, 1998; and Leisten et al., Clin. Immunol. Immunopathol 56:108-115, 1990), and anti-IL-6 antibodies have been reported for treatment of arthritis (Wendling et al., J Rheumatol. 20:259-262, 1993). In addition, estrogen has been shown to induce suppression of experimental autoimmune encephalomyelitis and collagen-induced arthritis in mice (Jansson et al., Neuroimmunol. 53:203-207, 1994).

The cytokine IL-6 has also been shown to be an important factor in inducing the formation of osteoclasts (Girasole et al., supra; Jilka et al. (1992), supra; Jilka et al. (1995), supra; Kimble et al. (1995), supra; Pacifici et al., supra; and Passeri et al., supra). Other investigators have shown that administration of the neutralizing antibody, antisense oligos, or the Sant 5 antagonist against IL-6, reduces the number of osteoclasts in trabecular bone of ovariectomized mice (Devlin et al., J. Bone Miner 13:393-399, 1998; Girasole et al., supra; Jilka et al. (1992), supra; and Schiller et al., Endocrinology 138:4567-4571, 1997), the ability of human giant cells to resorb dentine (Ohsaki et al., Endocrinology 131:2229 2234, 1993; and Reddy et al., J. Bone Min. Res. 9:753-757, 1994), and the formation of osteoclasts in normal human bone marrow culture. It has also been found that estrogen downregulates the IL-6 promoter activity by interactions between the estrogen receptor and the transcription factors NF-κB and C/EBPβ (Stein et al., Mol. Cell Biol. 15:4971-4979, 1995).

Granulocyte-macrophage colony-stimulating factor (GM-CSF) has been suggested to play a role in the proliferation of osteoclastic precursor cells. In long term cultures of human or mouse bone marrow cells or peripheral blood cells, GM-CSF promotes the formation of osteoclastic cells (Kurihara et al., Blood 74:1295-1302, 1989; Lorenzo et al., J. Clin. Invest. 80:160-164, 1987; MacDonald et al., J Bone Miner 1:227-233, 1986; and Shinar et al, Endocrinology 126:1728-1735, 1990). Bone marrow cells isolated from postmenopausal women, or women who discontinued estrogen therapy, expressed higher levels of GM-CSF than cells from premenopausal women (Bismar et al., J. Clin. Endocrinol. Metab. 80:3351-3355, 1995). Expression of GM-CSF has also been shown to be associated with the tissue distribution of bone-resorbing osteoclasts in patients with erosion of orthopedic implants (Al-Saffar et al., Anatomic Pathology 105:628-693, 1996).

As noted above, it had previously been assumed that estrogen binds to a single estrogen receptor (ER) in cells, causing conformational changes that result in release from heat shock proteins and binding of the receptor as a dimer to the so-called estrogen response element in the promoter region of a variety of genes. Further, pharmacologists have generally believed that non-steroidal small molecule ligands compete for binding of estrogen to ER, acting as either antagonists or agonists in each tissue where the estrogen receptor is expressed. Thus, such ligands have traditionally been classified as either pure antagonists or agonists. This is no longer believed to be correct.

Rather, it is now known that estrogen modulates cellular pharmacology through gene expression, and that the estrogen effect is mediated by estrogen receptors. As noted above, there are currently two estrogen receptors, ER-α and ER-β. The effect of estrogen receptor on gene regulation can be mediated by a direct binding of ER to the estrogen response element (ERE)—Aclassical pathway@ (Jeltsch et al., Nucleic Acids Res. 15:1401-1414, 1987; Bodine et al., Endocrinology 139:2048-2057, 1998), binding of ER to other transcription factors such as NF-κB, C/EBP-β or AP-1-Anon-classical pathway@ (Stein et al., Mol. Cell Biol. 15:4971-4979, 1995; Paech et al., Science 277:1508-1510, 1997; Duan et al., Endocrinology 139:1981-1990, 1998), and through non-genomic effects via extranuclear estrogen receptor signaling that potentially include plasma membrane ER (Nadal, A. et al., Trends in Pharmacological Sciences 22:597-599, 2001; Wyckoff, M. H. et al., J. Biol. Chem. 276: 27071-27076, 2001; Chung, Y-L. et al., Int. J. of Cancer 97:306-312, 2002; Kelly, M. J. et al., Trends Endocrinol. Metab. 10:369-374, 1999; Levin, E. R. et al., Trends Endocrinol. Metab. 10:374-377, 1999).

Progress over the last few years has shown that ER associates with co-activators (e.g., SRC-I, CBP and SRA) and co-repressors (e.g., SMRT and N-CoR), which also modulate the transcriptional activity of ER in a tissue-specific and ligand-specific manner. In such cases, ER interacts with the transcription factors critical for regulation of these genes. Transcription factors known to be modulated in their activity by ER include, for example, AP-1, NF-κB, C/EBP and Sp-1. In addition, orphan nuclear receptors, such as estrogen receptor-related receptors α, β, γ (ERR-α, ERR-β, ERR-γ), have been identified. Although estradiol does not appear to be a ligand for the ERRs, some SERMs and other traditional ER-ligands have been shown to bind to the receptors with high affinity (Coward, P. et al., Proc. Natl. Acad. Sci. 98:8880-8884, 2001; Lu, D. et al., Cancer Res. 61:6755-6761, 2001; Tremablay, G. B. et al., Endocrinology 142:4572-4575, 2001; Chen, S. et al., J. Biol. Chem. 276:28465-28470, 2001).

Furthermore, ER-α and ER-β have both overlapping and different tissue distributions, as analyzed predominantly by RT-PCR or in-situ hybridization due to a lack of good ER-β antibodies. Some of these results, however, are controversial, which may be attributable to the method used for measuring ER, the species analyzed (rat, mouse, human) and/or the differentiation state of isolated primary cells. Very often tissues express both ER-α and ER-β, but the receptors are localized in different cell types. In addition, some tissues (such as kidney) contain exclusively ER-α, while other tissues (such as uterus, pituitary and epidymis) show a great predominance of ER-α (Couse et al., Endocrinology 138, 4613-4621, 1997; Kuiper et al., Endocrinology 138, 863-870, 1997). In contrast, tissues expressing high levels of ER-β include prostate, testis, ovaries and certain areas of the brain (Brandenberger et al., J. Clin. Endocrinol. Metab. 83, 1025-8, 1998; Emnark et al., J. Clinic. Endocrinol. Metabol. 82, 4258-4265, 1997; Laflamme et al., J. Neurobiol. 36, 357-78, 1998; Sar and Welsch, Endocrinology 140, 963-71, 1999; Shughrue et al., Endocrinology 138, 5649-52, 1997a; Shughrue et al., J. Comp. Neurol. 388, 507-25, 1997b).

The development of ER-α (Korach, Science 266, 1524-1527, 1994) and ER-β (Krege et al., Proc. Natl. Acad. Sci. USA 95, 15677-82, 1998) knockout mice further demonstrate that ER-β has different functions in different tissues. For example, ER-α knockout mice (male and female) are infertile, females do not display sexual receptivity and males do not have typical male-aggressive behavior (Cooke et al., Biol. Reprod. 59, 470-5, 1998; Das et al., Proc. Natl. Acad. Sci. USA 94, 12786-12791, 1997; Korach, 1994; Ogawa et al., Proc. Natl. Acad. Sci. USA 94, 1476-81, 1997; Rissman et al., Endocrinology 138, 507-10, 1997a; Rissman et al., Horm. Behav. 31, 232-243, 1997b). Further, the brains of these animals still respond to estrogen in a pattern that is similar to that of wild-type animals (Shughrue et al., Proc. Natl. Acad. Sci. USA 94, 11008-12, 1997c), and estrogen still inhibits vascular injury caused by mechanical damage (Iafrati et al., Nature Med. 3, 545-8, 1997). In contrast, mice lacking the ER-β develop normally, are fertile and exhibit normal sexual behavior, but have fewer and smaller litters than wild-type mice (Krege et al., 1998), have normal breast development and lactate normally. The reduction in fertility is believed to be the result of reduced ovarian efficiency, and ER-β is the predominant form of ER in the ovary, being localized in the granulosa cells of maturing follicles.

In summary, compounds which serve as estrogen antagonists or agonists have long been recognized for their significant pharmaceutical utility in the treatment of a wide variety of estrogen-related conditions, including conditions related to the brain, bone, cardiovascular system, skin, hair follicles, immune system, bladder and prostate (Barkhem et al., Mol. Pharmacol. 54, 105-12, 1998; Farhat et al., FASEB J. 10, 615-624, 1996; Gustafsson, Chem. Biol. 2, 508-11, 1998; Sun et al., 1999; Tremblay et al., Endocrinology 139, 111-118, 1998; Turner et al., Endocrinology 139, 3712-20, 1998). In addition, a variety of breast and non-breast cancer cells have been described to express ER, and serve as the target tissue for specific estrogen antagonists (Brandenberger et al., 1998; Clinton and Hua, Crit. Rev. Oncol. Hematol. 25, 1-9, 1997; Hata et al., Oncology 55 Suppl 1, 35-44, 1998; Rohlff et al., Prostate 37, 51-9, 1998; Simpson et al., J. Steroid Biochem Mol Biol 64, 137-45,1998; Yamashita et al., Oncology 55 Suppl 1, 1998).

In recent years a number of both steroidal and nonsteroidal compounds which interact with ER have been developed. For example, Tamoxifen was originally developed as an anti-estrogen and used for the treatment of breast cancer, but more recently has been found to act as a partial estrogen agonist in the uterus, bone and cardiovascular system. Raloxifene is another compound that has been proposed as a SERM, and has been approved for treatment of osteoporosis.

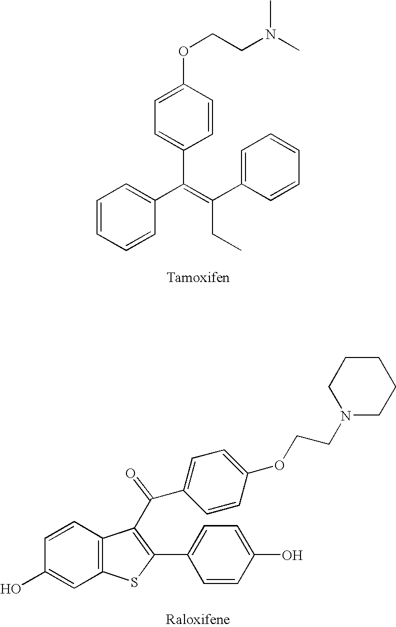

Analogs of Raloxifene have also been reported (Grese et al., J. Med. Chem. 40:146-167, 1997).

As for coumarin-based compounds, a number of structures have been proposed, including the following: Roa et al., Synthesis 887-888, 1981; Buu-Hoi et al., J. Org. Chem. 19:1548-1552, 1954; Gupta et al., Indian J. Exp. Biol. 23:638-640, 1985; Published PCT Application No. WO 96/31206; Verma et al., Indian J. Chem. 32B:239-243, 1993; Lednicer et al., J. Med. Chem. 8:725-726, 1965; Micheli et al., Steroids 5:321-335, 1962; Brandt et al., Int. J. Quantum Chemistry: Quantum Biol. Symposia 13:155-165, 1986; Wani et al., J. Med. Chem. 18:982-985, 1975; Pollard et al., Steroids 11:897-907, 1968.

Accordingly, there is a need in the art for compounds useful for treating a bone-resorbing disease, cancer, arthritis or an estrogen-related condition.

Citation or identification of any reference in Section 2 of this application is not to be construed as an admission that the reference is prior art to the present application.

### 3. SUMMARY OF THE INVENTION

The invention relates to compounds having the following general structure (I): 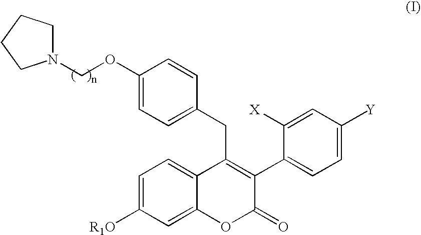
and pharmaceutically acceptable salts thereof, wherein:
n is 2, 3 or 4;
R₁ is hydrogen, C(═O)R₂, C(═O)OR₂, C(═O)NHR₂, C(═O)NR₂R₃, or S(═O₂)NR₂R₃;
R₂ and R₃ are independently C₁₋₈alkyl, C₆₋₁₂aryl, C₇₋₁₂arylalkyl, or a five- or six-membered heterocycle containing up to two heteroatoms selected from O, NR₄ and S(O)_{φ wherein each of the above groups are optionally substituted with one to three substituents independently selected from R}₅ and q is 0, 1 or 2;
R₄ is hydrogen or C₁₋₄ alkyl;
R₅ is hydrogen, halogen, hydroxy, C₁₋₆alkyl, C₁₋₄alkoxy, C₁₋₄acyloxy, C₁₋₄thio, C₁₋₄alkylsulfinyl, C₁₋₄alkylsulfonyl, (hydroxy)C₁₋₄alkyl, C₆₋₁₂aryl, C₇₋₁₂aralkyl, COOH, CN, CONHOR₆, SO₂NHR₆, NH₂, C₁₋₄alkylamino, C₁₋₄dialkylamino, NHSO₂R₆, NO₂, or a five- or six-membered heterocycle, where each occurrence of R₆ is independently C₁₋₆alkyl;
X is hydrogen, halogen or trifluoromethyl; and
Y is halogen or trifluoromethyl.

The invention also relates to a method of obtaining a compound of formula (I), wherein R₁ is H, by demethylation of a compound of formula (II).

The invention further relates to a method for inhibiting a cytokine in a patient, comprising administering to a patient in need thereof an effective amount of a compound of formula (I), (II) or a pharmaceutically acceptable salt of the compound.

The invention further relates to a method for treating or preventing a bone-resorbing disease in a patient, comprising administering to a patient in need thereof an effective amount of a compound of formula (I), (II) or a pharmaceutically acceptable salt of the compound.

The invention further relates to a method for treating or preventing cancer in a patient, comprising administering to a patient in need thereof an effective amount of a compound of formula (I), (II) or a pharmaceutically acceptable salt of the compound.

The invention further relates to a method for treating or preventing arthritis in a patient, comprising administering to a patient in need thereof an effective amount of a compound of formula (I), (II) or a pharmaceutically acceptable salt of the compound.

The invention further relates to a method for modulating gene expression in a cell expressing ER, comprising contacting the cell with an effective amount of a compound of formula (I), (II) or a pharmaceutically acceptable salt of the compound.

The invention further relates to a method for modulating gene expression in a tissue expressing ER, comprising contacting the cell with an effective amount of a compound of formula (I), (II) or a pharmaceutically acceptable salt of the compound.

The invention further relates to a method for activating the function of ER in a bone cell, comprising contacting bone a cell with an effective amount of a compound of formula (I), (II) or a pharmaceutically acceptable salt of the compound.

The invention further relates to a method for inhibiting the function of ER in a breast cancer cell, an ovarian cancer cell, an endometrial cancer cell, a uterine cancer cell, a prostate cancer cell or a hypothalamus cancer cell, comprising contacting the cell with an effective amount of a compound of formula (I), (II) or a pharmaceutically acceptable salt of the compound.

The invention further relates to a method for inhibiting the expression of IL-6 in a cell, comprising contacting a cell capable of expressing ER and IL-6 with an effective amount of a compound of formula (I), (II) or a pharmaceutically acceptable salt of the compound.

The invention further relates to methods for inhibiting proliferation of a cancer or neoplastic cell, comprising contacting a cancer or neoplastic cell capable of expressing ER with an effective amount of a compound of formula (I), (II) or a pharmaceutically acceptable salt of the compound.

The methods of the invention further comprise the administration of an effective amount of another therapeutic agent. Examples of other therapeutic agents include, but are not limited to, an agent useful for the treatment or prevention of an estrogen-related condition, an agent useful for the treatment or prevention of a bone-loss disease, an agent useful for the reduction of a patient=s serum cholesterol level and an agent useful for the treatment or prevention of cancer or a neoplastic disease.

The present invention may be understood more fully by reference to the detailed description and examples, which are intended to exemplify non-limiting embodiments of the invention.

### 4. DETAILED DESCRIPTION OF THE INVENTION

The invention relates to compounds of formula (I): 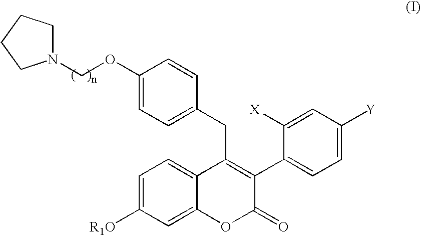
and pharmaceutically acceptable salts thereof, wherein:
n is 2, 3 or 4;
R₁ is hydrogen, C(═O)R₂, C(═O)OR₂, C(═O)NHR₂, C(═O)NR₂R₃, or S(═O₂)NR₂R₃;
R₂ and R₃ are independently C₁₋₈alkyl, C₆₋₁₂aryl, C₇₋₁₂arylalkyl, or a five- or six-membered heterocycle containing up to two heteroatoms selected from O, NR₄ and S(O)_{q}, wherein each of the above groups are optionally substituted with one to three substituents independently selected from R₅ and q is 0, 1 or 2;
R₄ is hydrogen or C₁₋₄ alkyl;
R₅ is hydrogen, halogen, hydroxy, C₁₋₆alkyl, C₁₋₄alkoxy, C₁₋₄acyloxy, C₁₋₄thio, C₁₋₄alkylsulfinyl, C₁₋₄alkylsulfonyl, (hydroxy)C₁₋₄alkyl, C₆₋₁₂aryl, C₇₋₁₂aralkyl, COOH, CN, C(═O)NHOR₆, S(═O₂)NHR₆, NH₂, C₁₋₄alkylamino, C₁₋₄dialkylamino, NHSO₂R₆, NO₂, or a five- or six-membered heterocycle, where each occurrence of R₆ is independently C₁₋₆alkyl;
X is hydrogen, halogen or trifluoromethyl; and
Y is halogen or trifluoromethyl.

In a preferred embodiment, the compounds of formula (I) are those wherein n=2 and R₁ is hydrogen.

The invention further relates to a method for obtaining compounds of formula (I), wherein R₁ is H, comprising the step of demethylating a compound of formula (II) shown below: 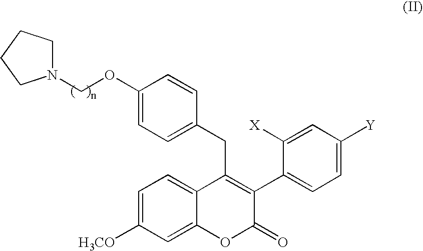
or a pharmaceutically acceptable salt thereof, wherein n is 2, 3 or 4 and X and Y are as defined above.

The demethylation of compounds of formula (II) can be achieved using any method known in the art useful in the deprotection of phenolic methyl ethers. Examples of such methods can be found in Greene, T. W., Protective Groups in Organic Synthesis, Chapter 3, John Wiley and Sons, New York, 1981, pp. 88-92, which is incorporated herein by reference in its entirety. Preferably, demethylation proceeds by a method comprising contacting a compound of formula (II) with about 1.0 to about 50.0 molar equivalents of a demethylating agent such as iodotrimethylsilane, pyridine hydrochloride, hydrobromic acid, hydrochloric acid, hydroiodic acid, a Grignard reagent, a Lewis acid or a strong nucleophile. More preferably, the demethylating agent is aqueous HBR, more preferably as a mixture in acetic acid. In a more preferred embodiment, demethylation is achieved by heating the compound of formula (II), or a pharmaceutically acceptable salt thereof, in the presence of the demethylating agent, optionally in the presence of a solvent, preferably a carboxylic acid, at a temperature of about room temperature to about 200 EC, preferably at a temperature of about 100 EC to about 160 EC for 15 minutes to about 24 hours. In one embodiment, the demethylation reaction vessel is sealed, for example a sealed tube, to prevent solvent evaporation, particularly where the boiling point of the solvent is lower than the temperature of the demethylation reaction. The acid salt of compounds of formula (I), wherein R₁ is H, can be obtained by isolating the compound directly from the demethylation reaction which can then be used to prepare the corresponding pharmaceutically acceptable salt. The free base form is available upon washing the acid salt with an appropriate base such as sodium hydroxide and isolating the compound.

The resulting compounds of formula (I), wherein R₁ is H, that are produced by demethylation of compounds of formula (II), are useful as cytokine inhibitors as well as for the treatment or prevention of a bone-resorbing disease, cancer, arthritis or an estrogen-related condition. The compounds of formula (I), wherein R₁ is H, that are produced by demethylation of compounds of formula (II) are also useful as intermediates in the synthesis of compounds of formula (I) wherein R₁ is C(═O)R₂, C(═O)OR₂, C(═O)NHR₂, C(═O)NR₂R₃, or S(═O₂)NR₂R₃.

The compounds of formula (I) and pharmaceutically acceptable salts thereof (collectively, the “benzopyranone compounds”), are useful for treating or preventing a bone-resorbing disease, cancer, arthritis or an estrogen-related condition. The benzopyranone compounds are also useful for inhibiting a cytokine in a patient and modulating gene expression in a cell and/or tissue expressing ER. Thus, the compounds of this invention may be administered as a therapeutic and/or prophylactic agent.

As used herein, a “C₆₋₁₂aryl” is an aromatic moiety containing from 6 to 12 carbon atoms. In one embodiment, the C₆₋₁₂aryl is selected from (but not limited to) phenyl, tetralinyl, and napthalenyl.

A “C₇₋₁₂aralkyl” is an arene containing from 7 to 12 carbon atoms, and has both aliphatic and aromatic units. In one embodiment, the C₇₋₁₂aralkyl is an aryl group bonded directly through an alkyl group, such as (but not limited to) benzyl, ethylbenzyl (i.e., —(CH₂)₂phenyl), propylbenzyl and isobutylbenzyl.

A “C₃₋₁₂heterocycle” is a compound that contains a ring made up of more than one kind of atom, and which contains 3 to 12 carbon atoms, including (but not limited to) pyrrolidinyl, pyrrolyl, indolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolidinyl, 2-oxazepinyl, azepinyl, 4-piperidonyl, pyridyl, N-oxo-pyridyl, pyrazinyl, pyrimidinyl, pyridazinyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolane and tetrahydro-1,1-dioxothienyl, dioxanyl, isothiazolidinyl, thietanyl, thiiranyl, triazinyl, and triazolyl.

A “C₄₋₁₆heterocyclealkyl” is a compound that contains a C₃₋₁₂heterocycle as listed above linked to a C₁₋₈alkyl.

A “C₁₋₈alkyl” is a straight chain or branched carbon chain containing from 1 to 8 carbon atoms, including (but not limited to) methyl, ethyl, n-propyl, n-butyl, n-pentyl, n-hexyl, and the like. Similarly, a “C₁₋ₓ alkyl” has the same meaning, but wherein “x” represents the number of carbon atoms less than eight, such as C₁₋₆alkyl.

A “substituted” C₁₋ₓalkyl, C₆₋₁₂aryl, C₇₋₁₂aralkyl, C₃₋₁₂heterocycle, or C₄₋₁₆heterocyclealkyl moiety is a C₁₋ₓalkyl, C₆₋₁₂aryl, C₇₋₁₂aralkyl, C₃₋₁₂heterocycle, or C₄₋₁₆heterocyclealkyl moiety having at least one hydrogen atom replaced with a substituent.

A “substituent” is a moiety selected from halogen, —OH, —R′, —OR′, —COOH, —COOR′, —COR′, —CONH₂, —NH₂, —NHR′, —NR′R′, —SH, —SR′, —SOOR′, —SOOH and —SOR′, where each occurrence of R′ is independently selected from an unsubstituted or substituted C₁₋₈alkyl, C₆₋₁₂aryl, C₇₋₁₂aralkyl, C₃₋₁₂heterocycle or C₄₋₁₆heterocyclealkyl.

A “halogen” is fluorine, chlorine, bromine or iodine.

The benzopyranone compounds can have chiral centers and can occur as racemates, racemic mixtures and as individual enantiomers or diastereomers. All such isomeric forms are included within the present invention, including mixtures thereof. Furthermore, some of the crystalline forms of the benzopyranone compounds can exist as polymorphs, which are included in the present invention. In addition, some of the benzopyranone compounds can also form solvates with water or other organic solvents. Such solvates are similarly included within the scope of this invention.

An estrogen “agonist” is a compound that binds to ER and mimics the action of estrogen in one or more tissues, while an “antagonist” binds to ER and blocks the action of estrogen in one or more tissues. Further, the term “estrogen-related condition” encompasses any condition associated with elevated or depressed levels of estrogen, a selective estrogen receptor modulator (SERM) or ER. In this context, ER includes both ER-α and/or ER-β, as well as any isoforms, mutations and proteins with significant homology to ER.

A “patient” is an animal, including, but not limited to, an animal such a cow, monkey, horse, sheep, pig, chicken, turkey, quail, cat, dog, mouse, rat, rabbit, and guinea pig, and is more preferably a mammal, and most, preferably a human.

Although not intending to be limited by the following theory, particularly in the context of bone-resorbing diseases, it is believed that the benzopyranone compounds function by blocking cytokine production and/or by inhibiting formation of osteoclasts.

The present invention also relates to pharmaceutical compositions comprising an effective amount of a benzopyranone compound and optionally a pharmaceutically acceptable carrier or vehicle, wherein a pharmaceutically acceptable carrier or vehicle can comprise an excipient, diluent, or a mixture thereof. Other embodiments of the present invention include methods for treating or preventing bone-resorbing diseases, including, but not limited to, osteoporosis, metastatic bone cancer and hypercalcemia, osteolytic lesions with orthopedic implants, Paget's disease, and bone loss associated with hyperparathyroidism; conditions associated with IL-6, including various cancers and arthritis; cancer, including breast cancer, prostrate cancer, colon cancer, endometrial cancer, multiple myeloma, renal cell carcinoma and cervical carcinoma; and arthritis, including adjuvant-, collagen-, bacterial- and antigen-induced arthritis, particularly rheumatoid arthritis. These methods comprise administering an effective amount of a benzopyranone compound to a patient in need thereof.

In addition, the benzopyranone compounds are useful for treating or preventing a wide range of estrogen-related conditions, including, but not limited to, breast cancer, osteoporosis, endometriosis, cardiovascular disease, hypercholesterolemia, prostatic hypertrophy, prostatic carcinomas, obesity, hot flashes, skin effects, mood swings, memory loss, prostate cancer, menopausal syndromes, hair loss (alopecia), type-II diabetes, Alzheimer's disease, urinary incontinence, GI tract conditions, spermatogenesis, vascular protection after injury, endometriosis, learning and memory, CNS effects, plasma lipid levels, acne, cataracts, hirsutism, other solid cancers (such as colon, lung, ovarian, melanoma, CNS, and renal), multiple myeloma, lymphoma, and adverse reproductive effects associated with exposure to environmental chemicals or natural hormonal imbalances.

The benzopyranone compounds are also useful for oral contraception; relief for the symptoms of menopause; prevention of threatened or habitual abortion; relief of dysmenorrhea; relief of dysfunctional uterine bleeding; relief of endometriosis; an aid in ovarian development; treatment of acne; diminution of excessive growth of body hair in women (hirsutism); the prevention or treatment of cardiovascular disease; prevention and treatment of atherosclerosis; prevention and treatment of osteoporosis; treatment of benign prostatic hyperplasia and prostatic carcinoma obesity; and suppression of post-partum lactation. The benzopyranone compounds also have a beneficial effect on plasma lipid levels and as such are useful in treating and preventing hypercholesterolemia. The benzopyranone compounds are further useful in the treatment and prevention of breast and ovarian cancer.

In another embodiment, the invention relates to a method for inhibiting a cytokine in a patient, comprising administering to a patient in need thereof an effective amount of a compound of formula (I), (II) or a pharmaceutically acceptable salt of the compound.

In a further embodiment, the invention relates to a method for modulating gene expression in a cell expressing ER, either ER-α r ER-β comprising contacting the cell with an effective amount of a compound of formula (I), (II) or a pharmaceutically acceptable salt of the compound.

In a further embodiment, the invention relates to a method for modulating gene expression in a tissue expressing ER, either ER-α or ER-β, comprising contacting the cell with an effective amount of a compound of formula (I), (II) or a pharmaceutically acceptable salt of the compound.

In a further embodiment, the invention relates to methods for activating the function of ER in a bone cell, comprising contacting a bone cell with an effective amount of a compound of formula (I), (II) or a pharmaceutically acceptable salt of the compound. Activating the function of ER in a bone cell is useful for treating or preventing osteoporosis.

In a further embodiment, the invention relates to methods for inhibiting the function of ER in a breast cancer cell, an ovarian cancer cell, an endometrial cancer cell, a uterine cancer cell, a prostate cancer cell or a hypothalamus cancer cell, comprising contacting the cell with an effective amount of a compound of formula (I), (II) or a pharmaceutically acceptable salt of the compound. Inhibiting the function of ER in a breast cancer cell, ovarian cancer cell, endometrial cancer cell, uterine cancer cell, prostate cancer cell or hypothalamus cancer cell is useful for inhibiting the growth of said cell and accordingly for treating or preventing cancer. In one embodiment, the breast cancer cell is MCF-7. In one embodiment, the ovarian cancer cell is BG-1.

In a further embodiment, the invention relates to methods for inhibiting the expression of IL-6 in a cell, comprising contacting a cell capable of expressing ER and IL-6 with an effective amount of a compound of formula (I), (II) or a pharmaceutically acceptable salt of the compound. In one embodiment, the cell that expresses ER and IL-6 is a bone cell. In another embodiment, the cell the expresses ER and IL-6 is a human U-2 OS osteosarcoma cell stably transfected with human ER-α. Inhibiting the expression of IL-6 in a cell in vivo is useful for the treatment of a bone-loss disease or bone cancer. In one embodiment, the bone-loss disease is osteoporosis. Inhibiting the expression of IL-6 in a cell in vitro is useful in a biological activity screening assay (e.g., as a standard) for the screening of a compound that inhibits the expression of IL-6.

In a further embodiment, the invention relates to methods for inhibiting cell proliferation of a cancer or neoplastic cell, comprising contacting a cancer or neoplastic cell capable of expressing ER with an effective amount of a compound of formula (I), (II) or a pharmaceutically acceptable salt of the compound. Examples of cancer or neoplastic cells capable of expressing ER include, but are not limited to, breast cells, ovarian cells, endometrial cells, uterine cells, prostate cells and hypothalamus cells. Inhibiting the proliferation of such cancer or neoplastic cells in vivo is useful for the treatment or prevention of cancer. Inhibiting the proliferation of such cancer or neoplastic cells in vitro is useful in a biological activity screening assay (e.g., as a standard) for anti-cancer or anti-neoplastic agents or in a diagnostic assay.

In a further embodiment, the invention involves methods for reducing a patient=s serum cholesterol level, comprising administering to a patient in need thereof an effective amount of a compound of formula (I), (II) or a pharmaceutically acceptable salt of the compound. The reduction of a patient's serum cholesterol level is useful for treating or preventing a cardiovascular disease or reducing the risk of cardiovascular disease.

In a further embodiment, the methods of the invention further comprise the administration of an effective amount of another therapeutic agent. In one embodiment, the other therapeutic agent is administered before, after or concurrently with the compound of formula (I), (II) or a pharmaceutically acceptable salt of the compound. In one embodiment, the time at which the compound of formula (I), (II) or a pharmaceutically acceptable salt of the compound exerts its therapeutic effect on the patient overlaps with the time at which the other therapeutic agent exerts its therapeutic effect on the patient.

In a further embodiment, the other therapeutic agent is useful for the treatment or prevention of an estrogen-related condition. Other therapeutic agents that are useful for the treatment or prevention of an estrogen-related condition include, but are not limited to, tamoxifen, raloxifene, medroxyprogesterone, danizol and gestrinone.

In a further embodiment, the other therapeutic agent is useful for the treatment or prevention of a bone-loss disease (e.g., osteoporosis). Other therapeutic agents useful for the treatment or prevention of a bone-loss disease include, but are not limited to, cathepsin K inhibitors (e.g., a pro-peptide of cathepsin K), bisphosphonates (e.g., eitodronate, pamidronate, alendronate, risedronate, zolendronate, ibandronate, clodronate or tiludronate), parathryoid hormone (APTH@) or fragments thereof, compounds that release endogenous PTH (e.g., a PTH releasing hormone) and calcitonin or fragments thereof.

In a further embodiment, the other therapeutic agent is useful for the reduction of a patient's serum cholesterol level. Other therapeutic agents useful for the reduction of a patient's serum cholesterol level include, but are not limited to, statins (e.g., lovastatin, atorvastatin, pravastatin) or a acyl-Coenzyme-A mimic.

In a further embodiment, the other therapeutic agent is useful for the treatment or prevention of cancer or a neoplastic disease (e.g., cancer of the breast, ovary, uterine, prostate or hypothalamus). Other therapeutic agents useful for the treatment or prevention of cancer or a neoplastic disease include, but are not limited to, alkylating agents (e.g., nitrosoureas), an anti-metabolite (e.g., methotrexate or hydroxyurea), etoposides, campathecins, bleomycin, doxorubicin, daunorubicin, colchicine, irinotecan, camptothecin, cyclophosphamide, 5-fluorouracil, cisplatinum, carboplatin, methotrexate, trimetrexate, erbitux, thalidomide, taxol, a vinca alkaloid (e.g., vinblastine or vincristine) or a microtubule stabilizer (e.g., an epothilone).

Further illustrative examples of therapeutic agents useful for the treatment or prevention of cancer include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; ImiDs; interleukin II (including recombinant interleukin II, or rIL2), interferon-2a; interferon alpha-2b; interferon alpha-n1; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; SelCid; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; temozolomide; temodar; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other therapeutic agents useful for the treatment or prevention of cancer include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); cell-cycle inhibitors (e.g., flavopiridol A, tryprostatin B, p19ink4D); cyclin-dependent kinase inhibitors (e.g., roscovitine, olomucine and purine analogs); MAP kinase inhibitors (CNI-1493); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; retinoic acid (e.g., 9-cis RA); histone deacetylase inhibitors (e.g., sodium butyrate, suberoylanilide hydroxamic acid); TRAIL; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin.

The benzopyranone compounds can be prepared according to the general reaction schemes (Route 1 and Route 2) shown below. 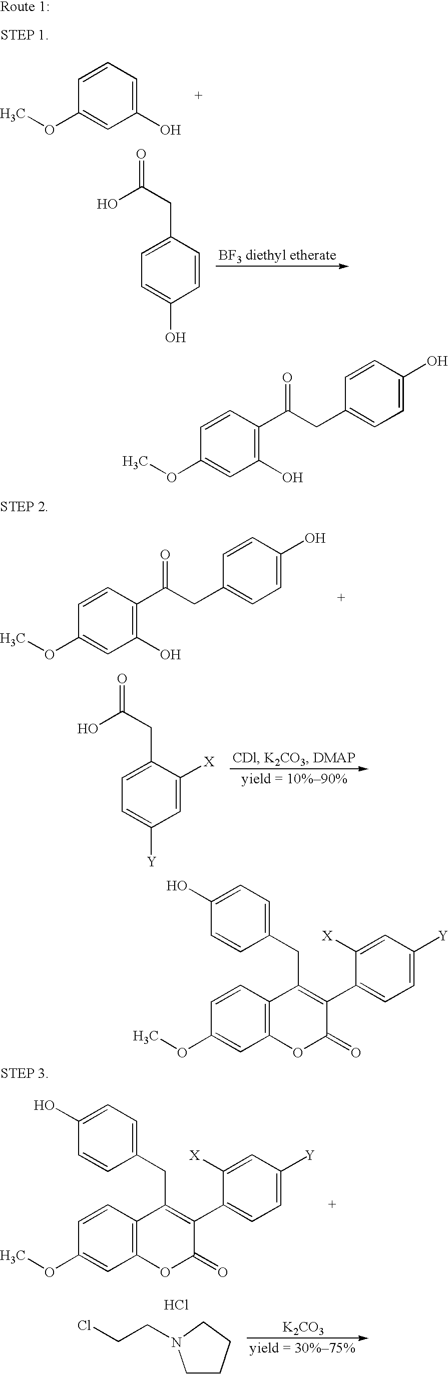 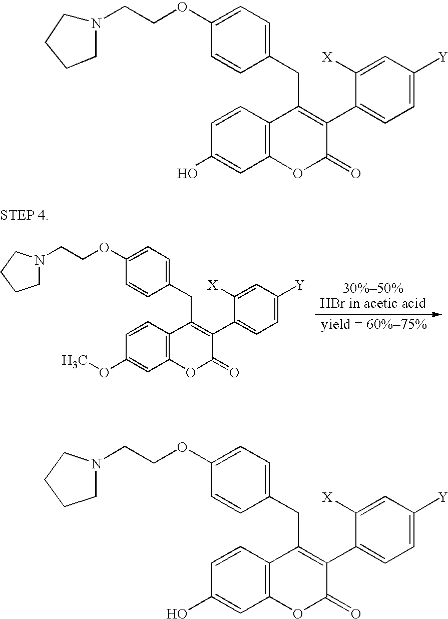
Step 1: Fries Reaction

Reaction yields are 40% to 55% and the reaction has been run on gram to multiple kilogram scale. On smaller scale reactions POCl₃ (solvent) and ZnCl₂ have been used in place of the BF₃ diethyl etherate.

Step 2: Coumarin Formation Reaction Summary

Reaction yields are typically 10% to 90% and the reactions have been run on a multiple gram scale. Powdered K₂CO₃ is essential for efficient reaction. Reactions have also been run by adding all reagents simultaneously instead of preactivating the acid as described above. Under these conditions slightly lower yields are obtained.

Step 3: Side-Chain Introduction Reaction Summary

Reaction yields are typically 30% to 70% and the reactions have been run on multiple gram scale. Powdered K2CO3 is essential and granular material results in incomplete or prolonged reaction times. The reaction yield in the examples provided are our most recent efforts and the yields were lower than expected. In the case of the dichloro analog, product precipitated on the column during flash chromatography In general this is the highest yielding step of the reaction sequence. The side-chain has also been introduced as described in the alternative synthesis scheme

Step 4: Demethylation Reaction Summary

Reaction yields are typically 60% to 75%. Sealed tube reaction minimizes HBr escape and greatly facilitates the reaction rate. Reactions run at atmospheric pressure require one day or more for completion.

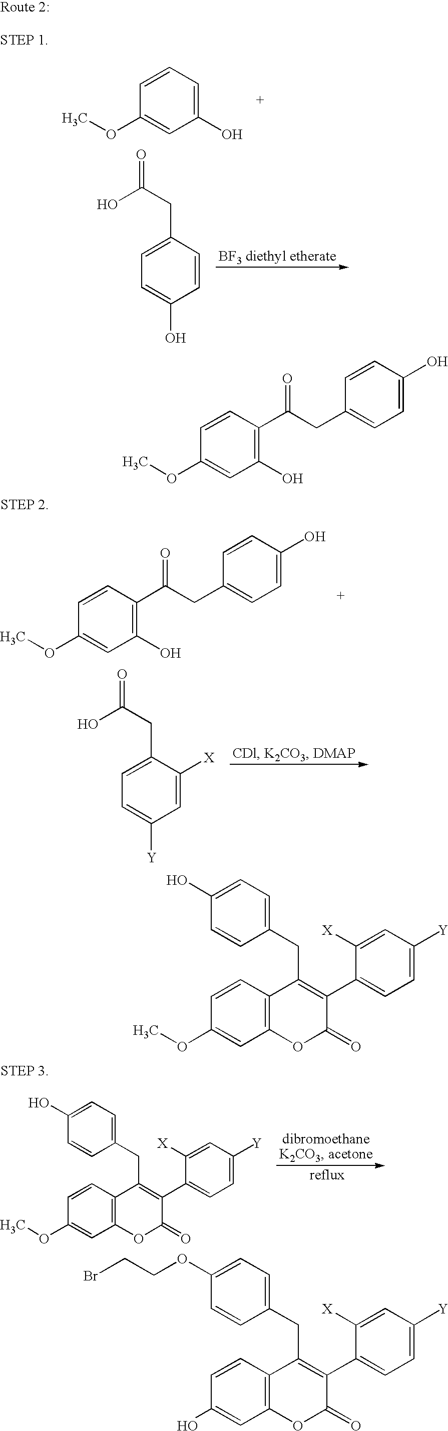

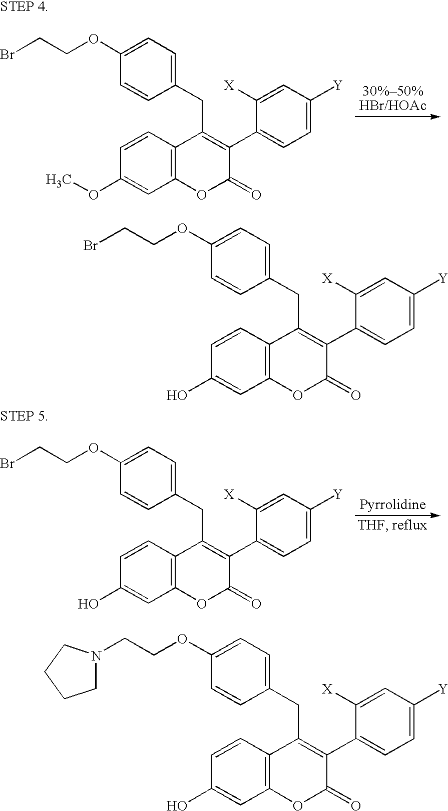

Methods of this invention involve administering an effective amount of a benzopyranone compound, or a pharmaceutical composition containing one or more of the same, to a patient in need thereof in an amount sufficient to treat the disease or condition of interest. To that end, the term “treat” (or the related terms “treating” and “treatment”) means administration of a compound, typically in combination with an appropriate delivery vehicle or agent, to a patient that does not show signs of a disease or condition (e.g., prophylactic or preventative administration) or that does show signs of a disease or condition (e.g., curative or treatment administration). Further, the phrase “effective amount” means a benzopyranone compound dose, or other active agent dose, that, after a given time, results in the desired effect. For example, in the context of bone-resorbing disease, an effective amount results in bones mass that is statistically different from that of animals treated with placebo. Similarly, for cancer and arthritis, an effective amount is an amount sufficient to produce the desired effect on the cancerous or arthritic tissue. In one embodiment, the “effective amount” is a dose capable of: treating or preventing a bone-resorbing disease; treating or preventing cancer; treating or preventing arthritis; modulating gene expression in a cell or tissue expressing ER; activating the function of ER in a bone cell; inhibiting the function of ER in a breast cancer cell, an ovarian cancer cell, an endometrial cell, a uterine cell, a prostate cell or a hypothalamus cell; inhibiting the function of ER in a cell that expresses ER and IL-6; inhibiting cell proliferation in a cancer or neoplastic cell; or reducing a patient's serum cholesterol level.

The benzopyranone compounds can exist as a pharmaceutically acceptable salt of a compound of structure (I) or (II). The pharmaceutically acceptable acid addition salts of the benzopyranone compounds can be formed of the compound itself, or of any of its esters, and include the pharmaceutically acceptable salts which are often used in pharmaceutical chemistry. For example, salts may be formed with organic or inorganic acids. Suitable organic acids include maleic, fumaric, benzoic, ascorbic, succinic, methanesulfonic, benzenesulfonic, toluenesulfonic, acetic, oxalic, trifluoroacetic, propionic, tartaric, salicylic, citric, gluconic, lactic, mandelic, cinnamic, aspartic, stearic, palmitic, formic, glycolic, glutamic, and benzenesulfonic acids. Suitable inorganic acids include hydrochloric, hydrobromic, sulfuric, phosphoric, and nitric acids. Additional salts include chloride, bromide, iodide, bisulfate, acid phosphate, isonicotinate, lactate, acid citrate, oleate, tannate, pantothenate, bitartrate, gentisinate, gluconate, glucaronate, saccharate, ethanesulfonate, p-toluenesulfonate, and pamoate (i.e., 1,1′-methylene-bis-(2-hydroxy-3-naphthoate)) salts. The term “pharmaceutically acceptable salt” is intended to encompass any and all acceptable salt forms.

Pharmaceutically acceptable salts can be formed by conventional and known techniques, such as by reacting a compound of this invention with a suitable acid as disclosed above. Such salts are typically formed in high yields at moderate temperatures, and often are prepared by merely isolating the compound from a suitable acidic wash in the final step of the synthesis. The salt-forming acid may dissolved in an appropriate organic solvent, or aqueous organic solvent, such as an alkanol, ketone or ester. On the other hand, if the benzopyranone compound is desired in the free base form, it may be isolated from a basic final wash step, according to known techniques. For example, a typical technique for preparing hydrochloride salt is to dissolve the free base in a suitable solvent, and dry the solution thoroughly, as over molecular sieves, before bubbling hydrogen chloride gas through it.

The benzopyranone compounds can be administered to a patient orally or parenterally in the conventional form of preparations, such as capsules, microcapsules, tablets, granules, powder, troches, pills, suppositories, injections, suspensions and syrups. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the benzopyranone compound in the pharmaceutical composition may be at a level that will exercise the desired effect; for example, about 0.1 mg to 100 mg in unit dosage for both oral and parenteral administration.

The benzopyranone compound can be usually administered one to four times a day with a unit dosage of 0.1 mg to 100 mg in human patients, but the above dosage may be properly varied depending on the age, body weight and medical condition of the patient and the type of administration. A preferred dose is 0.25 mg to 25 mg in human patients. One dose per day is preferred.

The dose of a benzopyranone compound to be administered to a human is rather widely variable and subject to the judgment of the attending physician. It should be noted that it may be necessary to adjust the dose of a benzopyranone compound when it is administered in the form of a salt, such as a laureate, the salt forming moiety of which has an appreciable molecular weight. The general range of effective administration rates of the benzopyranone compounds is from about 0.05 mg/day to about 100 mg/day. A preferred rate range is from about 0.25 mg/day to 25 mg/day. Of course, it is often practical to administer the daily dose of a benzopyranone compound in portions, at various hours of the day. However, in any given case, the amount of benzopyranone compound administered will depend on such factors as the solubility of the active component, the formulation used and the route of administration.

It is usually preferred to administer a benzopyranone compound orally for reasons of convenience. However, the benzopyranone compounds may equally effectively be administered percutaneously, or as suppositories for absorption by the rectum, if desired in a given instance.

The benzopyranone compounds can be administered as pharmaceutical compositions. The compositions can be in the form of tablets, chewable tablets, capsules, solutions, parenteral solutions, troches, suppositories and suspensions. Compositions can be formulated to contain a daily dose, or a convenient fraction of a daily dose, in a dosage unit, which may be a single tablet or capsule or convenient volume of a liquid.

The compositions can be readily formulated as tablets, capsules and the like; it is preferable to prepare solutions from water-soluble salts, such as the hydrochloride salt. In general, all of the compositions are prepared according to known methods in pharmaceutical chemistry. Capsules are prepared by mixing the benzopyranone compound with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours and similar edible powders.

Tablets are prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the die. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a benzopyranone compound as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the benzopyranone compounds can be delayed or prolonged by proper formulation. For example, a slowly soluble pellet of the benzopyranone compound can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the benzopyranone compound in oily or emulsified vehicles that allow it to disperse slowly in the serum.

### 5. EXAMPLES

The following Examples are presented by way of illustration, not limitation.

### Example 1

### 3-(2-chloro-4-trifluoromethylphenyl)-7-hydroxy-4-(4-(2-pyrrolidin-1-yl-ethoxy)-benzyl)-chromen-2-one

### A. (2-Chloro-4-trifluoromethylphenyl)-acetic acid

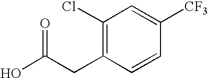

A solution of LiHMDS in toluene was prepared by the addition of n-BuLi (357 mL of a 1.6 M solution in hexanes, 571 mmol) to a cold (−78° C.) solution of HMDS (120.5 mL, 571 mmol) in toluene (700 mL). After 30 min, the reaction mixture was allowed to warm up to 10° C. over 1 h. The solution was then transferred via a cannula to a flame-dried, three-neck flask under N₂ containing Pd₂ dba₃ (4.18 g, 4.57 mmol) and (2′-dicyclohexylphosphanylbiphenyl-2-yl)-dimethylamine (3.77 g, 9.59 mmol). The mixture was stirred for 15 min at 15° C., cooled to −1° C. and t-butylacetate (70.5 mL, 525.4 mmol) was added dropwise. After 10 min, 3-chloro-4-iodobenzotrifluoride (70 g, 228.4 mmol) was added and the reaction mixture was warmed up to 28° C. After stirring at this temperature for 1.5 h, the mixture was filtered through silica gel, using toluene as eluent, and the solvent was removed in vacuo. The residue was purified using flash chromatography (silica gel, 98:2 hexanes:EtOAc) to yield (2-chloro-4-trifluoromethylphenyl)-acetic acid tert-butyl ester as a solid.

A solution of (2-chloro-4-trifluoromethylphenyl)-acetic acid tert-butyl ester (40 g, 135.7 mmol) in dioxane (100 mL) containing conc. HCl (31.3 mL) was stirred at 50° C. for 5 h. After cooling the mixture to r.t., it was diluted with Et₂O and the organic layer was washed with H₂O (3×). The organic phase was dried (MgSO₄) and the solvent was removed in vacuo. Recrystallization of the residue with AcOEt-hexane yielded the title compound as a solid showing: ¹H NMR (400 MHz, CDCl₃) δ 7.71 (s, 1H), 7.55 (dd, 1H, J=1.0, 8.0 Hz), 7.47 (d, 1H, J=8.0 Hz), 3.92 (s, 2H). MS (ESI) m/z 237 (M−H)⁻.

### B. 3-(2-Chloro-4-trifluoromethylphenyl)-4-(4-hydroxybenzyl)-7-methoxy-chromen-2 one

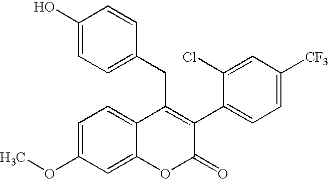

A mixture of 2-(chloro-4-trifluoromethylphenyl)-acetic acid (3.2 g, 13.41 mmol) and 1,1′-carbonyldiimidazole (2.72 g, 16.77 mmol) in DMF (15 mL) was heated to 70° C. for 25 min. The reaction mixture was cooled to 10° C. and K₂CO₃ (2.78 g, 20.1 mmol), 1-(2-hydroxy-4-methoxyphenyl)-2-(4-hydroxyphenyl)-ethan-1-one (1.73 g, 6.7 mmol, prepared as described in Example 4A), DMAP (164 mg, 1.34 mmol) and DMF (10 mL) were added. After stirring the reaction mixture at 115° C. for 1.5 h, the resulting suspension was cooled to r.t., poured onto AcOEt/H₂O and the layers were separated. The organic layer was washed with H₂O, aq 1N HCl and brine, was dried (MgSO₄) and the solvent was removed in vacuo. The resultant red residue was purified using flash chromatography (silica gel, 2:1 to 1:2 hexanes:Et₂O) to provide the title compound as a white solid showing: ¹H NMR (300 MHz, CDCl₃) δ 7.75 (s, 1H), 7.53 (br d, 1H, J=8.0 Hz), 7.47 (d, 1H, J=9.0 Hz), 7.33 (d, 1H, J=8.0 Hz), 6.92-6.85 (m, 3H), 6.80 (d, 1H, J=2.5, 9.0 Hz), 6.70 (d, 2H, J=8.5 Hz), 4.95-4.64 (very br s, 1H), 4.02 (d, 1H, J=15.5 Hz), 3.88 (s, 3H), 3.76 (d, 1H, J=15.5 Hz). MS (ESI) m/z 461 (M+H)⁺.

### C. 3-(2-Chloro-4-trifluoromethylphenyl)-7-methoxy-4-(4-(2-pyrrolidin-1-YI-ethoxy)-benzyl)-chromen-2-one

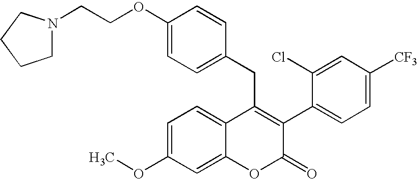

A mixture of 3-(2-chloro-4-trifluoromethylphenyl)-4-(4-hydroxybenzyl)-7-methoxychromen-2-one (460 mg, 1 mmol), 1-(2-chloroethyl)pyrrolidine hydrochloride (254.7 mg, 1.5 mmol) and K₂CO₃ (413.9 mg, 2.99 mmol) in EtOH (5 mL) was stirred for 2 min prior to the addition of H₂O (0.5 mL). The mixture was stirred at 55° C. for 2.5 h, after which time it was cooled to r.t. and poured into CHCl₃-H₂O. The layers were separated and the aqueous phase was extracted with CHCl₃ (3×). The combined organic layers were washed with brine, dried (MgSO₄) and the solvent was removed in vacuo. The resultant brown foam was purified using flash chromatography (silica gel, 19:1 CH₂Cl₂:MeOH) to provide the title compound as a pale brown foam which displayed: ¹H NMR (300 MHz, CD₃OD) δ 7.83 (s, 1H), 7.65 (d, 1H, J=9.0 Hz), 7.63 (d, 1H, J=8.0 Hz), 7.51 (d, 1H, J=8.0 Hz), 7.00 (d, 1H, J=2.5 Hz), 6.95 (d, 2H, J=8.5 Hz), 6.89 (dd, 1H, J=2.5, 9.0 Hz), 6.79 (d, 2H, J=8.5 Hz),), 4.07 (d, 1H, J=15.5 Hz), 4.05 (t, 2H, J=5.5 Hz), 3.90 (s, 3H), 3.83 (d, 1H, J=15.5 Hz), 2.89 (t, 2H, J=5.5 Hz), 2.72-2.60 (m, 4H), 1.90-1.75 (m, 4H) MS (ESI) m/z 558 (M+H)⁺.

### D. 3-(2-Chloro-4-trifluoromethylphenyl)-7-hydroxy-4-(4-(2-pyrrolidin-1-yl-ethoxy)-benzyl)-chromen-2-one

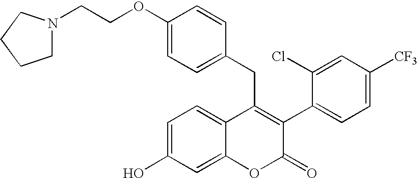

3-(2-Chloro-4-trifluoromethylphenyl)-7-methoxy-4-(4-(2-pyrrolidin-1-yl-ethoxy)-benzyl)-chromen-2-one (330 mg, 0.59 mmol) was dissolved in AcOH (2.4 mL)-48% aq HBr (2.4 mL). The mixture was stirred at 130° C. for 15 h. After cooling the mixture to r.t., it was poured onto EtOAc/aq NaHCO₃. 1M aq NaOH was then added to bring the pH to 8. The layers were separated and the aqueous layer was back-extracted with EtOAc (3×). The combined organic layers were washed with brine, dried (MgSO4) and the solvent was removed in vacuo. The residue was purified using flash chromatography (silica gel, 5:1 CH2Cl2-MeOH) to provide the title compound as a yellow foam showing: IR (KBr) v=3670-2140, 1709, 1611, 1569, 1511, 1367, 1323, 1247, 1172, 1133, 1081, 1067, 1044, 1012 cm-1. 1H NMR (400 MHz, CD₃OD) δ7.81 (d, 1H, J=1.5 Hz), 7.61 (dd, 1H, J=1.5, 8.0 Hz), 7.56 (d, 1H, J=8.8 Hz), 7.48 (d, 1H, J=8.0 Hz), 6.93 (d, 2H, J=8.5 Hz), 6.79 (d, 2H, J=8.5 Hz), 6.76 (d, 1H, J=2.5 Hz), 6.73 (dd, 1H, J=2.5, 8.8 Hz), 4.09 (t, 2H, J=5.5 Hz), 4.05 (d, 1H, J=15.5 Hz), 3.80 (d, 1H, J=15.5 Hz), 3.04 (t, 2H, J=5.5 Hz), 2.86-2.78 (m, 4H), 1.92-1.82 (m, 4H). HRMS (ESI) calcd for C₂₉H₂₅ClF3NO4 (M+H)+: 544.1502; found: 544.1504.

### Example 2

### 3-(4-chloro-2-trifluoromethylphenyl)-7-hydroxy-4-(4-(2-pyrrolidin-1-yl-ethoxy)-benzyl)-chromen-2-one

### A. (4-Chloro-2-trifluoromethylphenyl)-acetic acid

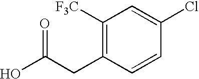

A solution containing 4-chloro-1-iodo-2-trifluoromethylbenzene (14.98 g, 48.9 mmol), Bu₃SnCH═CH₂ (15.7 mL, 53.7 mmol) and (Ph₃P)₄Pd (2.26 g, 1.955 mmol) in anhyd toluene (200 mL) was deoxygenated using vacuum-N₂ flush (3×). After refluxing the reaction mixture for 17 h, it was cooled to 0° C. and a solution of disiamylborane-methyl sulfide complex in toluene (˜1.95 M, 47 mL) was added dropwise over a period of ˜5 min. The disiamylborane-methyl sulfide complex solution was prepared by adding 2-methyl-2-butene (26 mL, 245 mmol) to a cold (0° C.) solution of borane-methyl sulfide complex (11.6 mL, 122.3 mmol) in anhyd toluene (25 mL) and stirring the resultant mixture at r.t. for 2 h. The bath was removed and the reaction mixture was stirred at r.t. for 3 h. After that period of time, the mixture was cooled to 0° C., EtOH (75 mL) was added slowly, followed by 2 N aq NaOH (37.5 mL) and 30% aq H₂O₂ (30 mL). The solution was stirred at r.t. for 1.5 h and was then poured onto Et₂O—H₂O. The layers were separated and the organic layer was washed with H₂O and brine, was dried (MgSO₄) and the solvent was removed in vacuo, while the bath temperature was maintained below 30° C. The black residue was purified using flash chromatography (silica gel; 4:1 to 3:1 hexanes:AcOEt) to afford 2-(4-chloro-2-trifluoromethyl-phenyl)-ethanol as a brown oil which was used directly in the next step.

To a solution of 2-(4-chloro-2-trifluoromethylphenyl)-ethanol in acetone (50 mL) at 0° C. was added dropwise a solution of Jones reagent (40.3 mL of a 2.67 M solution in H₂SO₄). After 25 min, the mixture was poured onto Et₂O/H₂O and the layers were separated. The organic layer was washed with H₂O and brine, dried (MgSO₄) and the solvent was removed in vacuo. The resultant orange solid was crystallized from hexane and heptane to furnish the title compound as a solid which showed: ¹H NMR (300 MHz, CDCl₃) δ 7.67 (d, 1H, J=2.0 Hz), 7.51 (dd, 1H, J=2.0, 8.0 Hz), 7.34 (d, 1H, J=8.0 Hz), 3.84 (s, 2H).

### B. 3-(4-Chloro-2-trifluoromethylphenyl)-4-(4-hydroxybenzyl)-7-methoxy-chromen-2-one

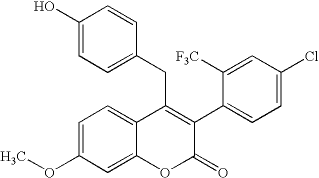

This compound was prepared using the methodology described above in Example 1B. The resultant residue was purified using flash chromatography (silica gel, 1:1 to 55:45 to 3:2 Et₂O:hexanes) to provide the title compound as a beige solid showing: ¹H NMR (300 MHz, CDCl₃) δ 7.76 (d, 1H, J=1.5 Hz), 7.50 (dd, 1H, J=1.5, 8.0 Hz), 7.41 (d, 1H, J=9.0 Hz), 7.15 (d, 1H, J=8.0 Hz), 6.89 (d, 1H, J=2.5 Hz), 6.85 (d, 2H, J=8.5 Hz), 6.77 (dd, 1H, J=2.5, 9.0 Hz), 6.70 (d, 2H, J=8.5 Hz), 4.00 (d, 1H, J=15.5 Hz), 3.87 (s, 3H), 3.61 (d, 1H, J=15.5 Hz). MS (ESI) m/z 461 (M+H)⁺.

### C. 3-(4-Chloro-2-trifluoromethylphenyl)-7-methoxy-4-(4-(2-pyrrolidin-1-yl-ethoxy)-benzyl)-chromen-2-one

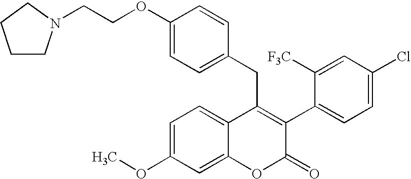

This compound was prepared using the methodology described above in Example 1C. The resultant brown foam was purified using flash chromatography (silica gel, 94:6 CH₂Cl₂:MeOH) to provide the title compound as a light yellow foam which displayed: ¹H NMR (300 MHz, CD₃OD) δ 7.83 (d, 1H, J=1.7 Hz), 7.61 (dd, 1H, J=1.7, 8.5 Hz), 7.55 (d, 1H, J=9.0 Hz), 7.34 (d, 1H, J=8.5 Hz), 7.00-6.93 (m, 3H), 6.85 (dd, 1H, J=2.5, 9.0 Hz), 6.81 (d, 2H, J=9.0 Hz), 4.11 (d, 1H, J=15.5 Hz), 4.05 (t, 2H, J=5.5 Hz), 3.88 (s, 3H), 3.61 (d, 1H, J=15.5 Hz), 2.90 (t, 2H, J=5.5 Hz), 2.71-2.61 (m, 4H), 1.86-1.77 (m, 4H). MS (ESI) m/z 558 (M+H)⁺.

### D. 3-(4-Chloro-2-trifluoromethylphenyl)-7-hydroxy-4-(4-(2-pyrrolidin-1-yl-ethoxy)-benzyl)-chromen-2-one

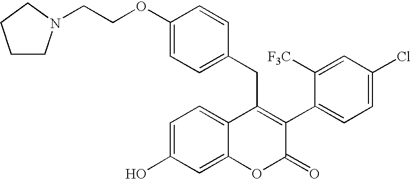

This compound was prepared using the methodology described above in Example 1D. The residue was purified using flash chromatography (silica gel, 5:1 CH₂Cl₂-MeOH) to provide the title compound as a yellow solid showing: IR (KBr) v=3700-2100, 1721, 1597, 1512, 1467, 1377, 1305, 1265, 1247, 1182, 1136, 1108, 1061, 1046, 1016, 843 cm-1. 1H NMR (400 MHz, CD3OD) δ 7.82 (d, 1H, J=2.0 Hz), 7.59 (dd, 1H, J=2.0, 8.0 Hz), 7.47 (d, 1H, J=9.0 Hz), 7.31 (d, 1H, J=8.0 Hz), 6.96 (d, 2H, J=8.5 Hz), 6.82 (d, 2H, J=8.5 Hz), 6.75 (d, 1H, J=2.5 Hz), 6.69 (dd, 1H, J=2.5, 9.0 Hz), 4.12-4.06 (m, 3H), 3.59 (d, 1H, J=15.5 Hz), 3.05 (t, 2H, J=5.5 Hz), 2.86-2.81 (m, 4H), 1.92-1.84 (m, 4H). HRMS (ESI) calcd for C₂₉H₂₅ClF₃NO₄ (M+H)⁺: 544.1502; found: 544.1505.

### Example 3

### 3-(2,4-bis-trifluoromethylphenyl)-7-hydroxy-4-(4-(2-pyrrolidin-1-yl-ethoxy)-benzyl)-chromen-2-one

### A. 3-(2,4-Bis-Trifluoromethylphenyl)-7-Hydroxy-4-(4-(2-Pyrrolidin-1-Y1-Ethoxy)-Benzyl)-Chromen-2-One

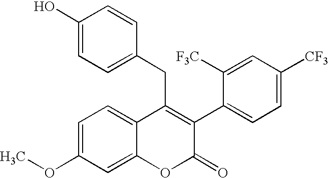

This compound was prepared using the methodology described above in Example 1B. The resultant residue was purified using flash chromatography (silica gel, 1:1 to 3:2 Et₂O:hexanes) to provide the title compound as a yellow foam showing: ¹H NMR (300 MHz, CDCl₃) δ 8.03 (s, 1H), 7.78 (d, 1H, J=8.0 Hz), 7.42 (d, 1H, J=8.8 Hz), 7.36 (d, 1H, J=8.0 Hz), 6.90 (d, 1H, J=2.5 Hz), 6.84 (d, 2H, J=8.5 Hz), 6.78 (dd, 1H, J=2.5, 8.8 Hz), 6.70 (d, 2H, J=8.5 Hz), 4.76 (s, 1H), 4.01 (d, 1H, J=16.0 Hz), 3.88 (s, 3H), 3.57 (d, 1H, J=16.0 Hz).

### B. 3-(2,4-bistrifluoromethylphenyl)-7-methoxy-4-(4-(2-pyrrolidin-1-yl-ethoxy)-benzyl)-chromen-2-one

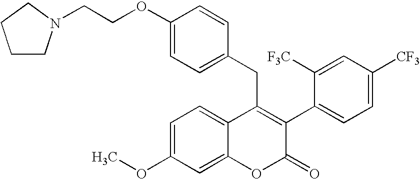

This compound was prepared using the methodology described above in Example 1C. The resultant brown foam was purified using flash chromatography (silica gel, 96:4 CH₂Cl₂:MeOH) to provide the title compound as a beige foam which displayed: ¹H NMR (300 MHz, CD₃OD) δ 8.09 (s, 1H), 7.93 (d, 1H, J=8.5 Hz), 7.59 (d, 1H, J=8.5 Hz), 7.57 (d, 1H, J=9.0 Hz), 6.99 (d, 1H, J=2.5 Hz), 6.97 (d, 2H, J=8.5 Hz), 6.87 (dd, 1H, J=2.5, 9.0 Hz), 6.81 (d, 2H, J=8.5 Hz), 4.13 (d, 1H, J=16.0 Hz), 4.05 (t, 2H, J=5.5 Hz), 3.89 (s, 3H), 3.59 (d, 1H, J=16.0 Hz), 2.89 (t, 2H, J=5.5 Hz), 2.73-2.58 (m, 4H), 1.87-1.77 (m, 4H). MS (ESI) m/z 592 (M+H)⁺.

### C. 3-(2,4-bistrifluoromethylphenyl)-7-hydroxy-4-(4-(2-pyrrolidin-1-ylethoxy)-benzyl)-chromen-2-one

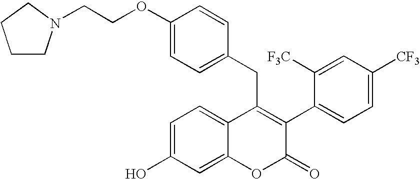

This compound was prepared using the methodology described above in Example 1D. The residue was purified using flash chromatography (silica gel, 3:1 CH₂Cl₂-MeOH) to provide the title compound as a yellow foam showing: IR (KBr) v=3700-2300, 1714, 1615, 1512, 1462, 1368, 1346, 1300, 1272, 1179, 1133, 1082, 1062, 1045, 1014, 846 cm-1. ¹H NMR (400 MHz, CD₃OD) δ 8.07 (s, 1H), 7.91 (br d, 1H, J=8.5 Hz), 7.56 (d, 1H, J=8.5 Hz), 7.49 (d, 1H, J=8.8 Hz), 6.96 (d, 2H, J=9.0 Hz), 6.82 (d, 2H, J=9.0 Hz), 6.77 (d, 1H, J=2.5 Hz), 6.71 (dd, 1H, J=2.5, 8.8 Hz), 4.11 (d, 1H, J=16.0 Hz), 4.11 (t, 2H, J=5.5 Hz), 3.57 (d, 1H, J=16.0 Hz), 3.08 (t, 2H, J=5.5 Hz), 2.90-2.83 (m, 4H), 1.92-1.86 (m, 4H). HRMS (ESI) calcd for C₃₀H₂₅F₆NO₄ (M+H)⁺: 578.1766; found: 578.1762.

### Example 4

### 3-(4-trifluoromethylphenyl)-4-(4-(2-pyrrolidin-1-yl-ethoxy)benzyl)-7-methoxychromen-2-one

### A. 1-(2-hydroxy-4-methoxyphenyl)-2-(4-hydroxyphenyl)ethan-1-one

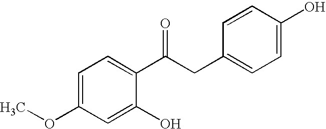

A suspension of 3-methoxyphenol (44.69 kg, 360 mol) and 4-hydroxyphenylacetic acid (68.5 kg, 450 mol) in 144 L of chlorobenzene was purged with nitrogen gas. Boron trifluoride diethyl etherate (177 L, 1440 mol) was added at 20 to 25° C. The suspension was heated to 80° C. and stirred for 4 to 5 h then cooled to 5 to 10° C. and stirred overnight.

The precipitated red/orange solid (undesired isomer) was filtered with N₂ pressure and the filtrate was quenched by pouring onto ice/H₂O. The filter cake was washed with CH₂Cl₂. The boron trifluoride etherate was quenched by the slow addition of 80% Na₂CO₃ (aq) until the pH of the aqueous solution reached 6 to 7. Gas evolution was observed and the product precipitated from solution.

The orange suspension was stirred at 20° C. overnight and subsequently filtered. The filter cake was washed with H₂O and MTBE and dried overnight to provide the desired product (38 kg, 42% yield, HPLC purity 95.1% a/a). 1H NMR (300 MHz, DMSO-d6) α12.30 (s, 1H), 9.31 (s, 1H), 7.99 (d, 1H, J=9.1 Hz), 7.08 (d, 2H, J=8.4 Hz), 6.70, (d, 2H, J=8.4 Hz), 6.53 (dd, 1H, J=2.5, 9.1 Hz), 6.47 (d, 1H, J=2.5 Hz), 4.18 (s, 2H), 3.81 (s, 3H). MS (ESI) m/z 259 (M+H)+.

### B. 3-(4-trifluoromethylphenyl)-4-(4-hydroxybenzyl)-7-methoxychromen-2-one

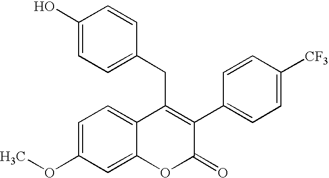

A solution of 4-trifluoromethylphenylacetic acid (15.2 g, 74.45 mmol) in 120 mL of DMF at 25° C. was treated with CDI (13.2 g, 82 mmol) in several portions over 5 minutes. The reaction mixture was warmed to 40° C. for 10 minutes then cooled to room temperature. 1-(2-hydroxy-4-methoxyphenyl)-2-(4-hydroxyphenyl)ethan-1-one (9.81 g, 38 mmol), K₂CO₃ (15.7 g, 114 mmol), and DMAP (0.93 g, 7.6 mmol) were added and the reaction mixture was warmed to 80° C. for 2 hours.

The suspension was cooled to room temperature and 200 mL of water was added. The aqueous layer was extracted with CH₂Cl₂ and the combined organic layer was dried (MgSO₄) then concentrated under vacuum. The resulting solid was purified using flash chromatography (CH₂Cl₂:EtOAc) to provide the desired product (10.2 g, 63%).

¹H NMR (300 MHz, DMSO-d6) 9.29 (s, 1H), 7.79 (d, 2H, J=8.7 Hz), 7.57 (d, 2H, J=8.7 Hz), 7.53 (d, 1H, J=8.5 Hz), 7.04 (d, 1H, J=2.3 Hz), 6.93 (d, 2H, J=8.9 Hz), 6.87 (dd, 1H, J=8.5, 2.3 Hz), 6.61 (d, 2H, J=8.9 Hz), 3.90 (s, 2H), 3.84 (s, 3H). MS (ESI) m/z 427 (M+H)⁺.

### C. 3-(4-trifluoromethylphenyl)-4-(4-(2-pyrrolidin-1-ylethoxy)benzyl)-7-methoxychromen-2-one

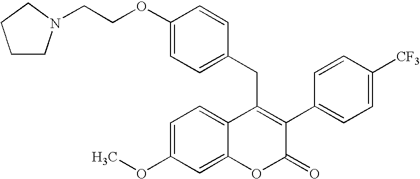

A solution of 3-(4-trifluoromethylphenyl)-4-(4-hydroxybenzyl)-7-methoxychromen-2-one (6.0 g, 14 mmol), 1-(2-chloroethyl)pyrrolidine hydrochloride (3.3 g, 22.5 mmol), and K₂CO₃ (6.6 g, 47.8 mmol) in 30 mL of DMF was warmed at 120° C. for 2 hours. Solvent was removed under reduced pressure. Water was added and the aqueous layer was extracted with ethyl acetate. The combined organic layer was dried and concentrated to provide a dark brown oil. Flash chromatography (CH₂Cl₂:EtOAc:MeOH:TEA) provided the desired product (4.7 grams, 64%). ¹H NMR (300 MHz, DMSO-d6) 7.79 (d, 2H, J=8.1 Hz), 7.58 (d, 2H, J=8.1 Hz), 7.51 (d, 1H, J=9.0 Hz), 7.08 (d, 2H, J=8.9 Hz), 7.06 (d, 1H, J=2.5 Hz), 6.87 (dd, 1H, J=2.5, 9.0 Hz), 6.82 (d, 2H, J=8.9 Hz), 4.08 (t, 2H, J=5.0 Hz), 3.96 (s, 2H), 3.84 (s, 3H), 3.17-3.12 (m, 2H), 2.94-2.88 (m, 4H), 1.83-1.78 (m, 4H). MS (ESI) m/z 524 (M+H)⁺.

### D. 3-(4-trifluoromethylphenyl)-4-(4-(2-pyrrolidin-1-yl-ethoxy)benzyl)-7-methoxychromen-2-one

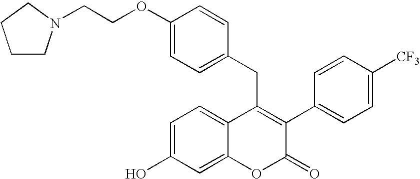

A solution of 3-(4-trifluoromethylphenyl)-4-(4-(2-pyrrolidin-1-yl-ethoxy)benzyl)-7-methoxychromen-2-one (4.2 grams, 8.02 mmol) and 25 mL of 30% HBr/HOAc in a sealed tube was warmed at 120° C. for 3 h. The solvent was removed under reduced pressure and the residue was quenched with NaHCO3 (aq). The aqueous layer was extracted with CH₂Cl₂ and the combined organic layer was concentrated. The crude product was purified by passage through a short column of silica gel followed by reverse phase preparative HPLC to provide the title compound (2.9 g, 71%). ¹H NMR (300 MHz, DMSO) 7.77 (d, 2H, J=8.0 Hz), 7.55 (d, 2H, J=8.0 Hz), 7.44 (d, 1H, J=8.8 Hz), 7.03 (d, 2H, J=8.0 Hz), 6.79 (d, 2H, 8.0 Hz), 6.76 (s, 1H), 6.70 (d, 1H, J=8.5 Hz), 3.97 (t, 2H, J=5.8 Hz), 3.92 (s, 2H), 2.72 (d, 2H, J=5.8 Hz), 2.50-2.47 (m, 4H), 1.66-1.64 (m, 4H). MS (ESI) m/z 510 (M+H)+.

### Example 5

### 3-(4-chlorophenyl)-4-(4-(2-pyrrolidin-1-yl-ethoxy)benzyl)-7-hydroxychromen-2-one hydrochloride

### A. 1-(2-hydroxy-4-methoxyphenyl)-2-(4-hydroxyphenyl)ethan-1-one

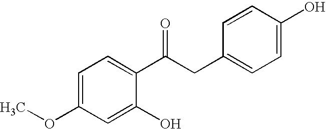

This compound was prepared using the methodology described above in Example 4A.

### B. 3-(4-chlorophenyl)-4-(4-hydroxybenzyl)-7-methoxychromen-2-one

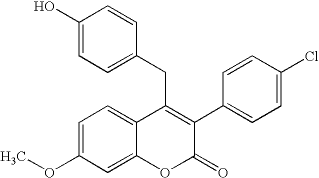

This compound was prepared using the methodology described above in Example 4B. ¹H NMR (300 MHz, DMSO-d6) 9.30 (s, 1H), 7.51 (d, 1H, J=9.1 Hz), 7.47 (d, 2H, J=8.2 Hz), 7.34 (d, 2H, J=8.2 Hz), 7.02 (d, 1H, J=2.2 Hz), 6.91 (d, 2H, J=8.5 Hz), 6.85 (dd, 1H, J=9.1, 2.2 Hz), 6.61 (d, 2H, J=8.5 Hz), 3.91 (s, 2H), 3.83 (s, 3H). MS (ESI) m/z 393 (M+H)⁺.

### C. 3-(4-chlorophenyl)-4-(4-(2-bromoethoxy)benzyl)-7-methoxychromen-2-one

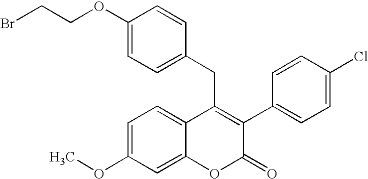

A solution of 3-(4-chlorophenyl)-4-(4-hydroxybenzyl)-7-methoxychromen-2-one (21.2 g, 54 mmol), dibromoethane (50.7 g, 270 mmol), and K₂CO₃ (8.3 g, 60 mmol) in 200 mL of acetone was heated at reflux for 12 h. The reaction mixture was cooled to room temperature and volatiles were removed under reduced pressure. Hexanes (500 mL) was added with stirring and the resulting solid that formed was collected by filtration. The material was rinsed with hexanes (2×100 mL), collected and dried under vacuum to provide the desired product (22.5 g, 83%). ¹H NMR (300 MHz, DMSO) 7.50 (d, 1H, J=9.1 Hz), 7.48 (d, 2H, J=8.2 Hz), 7.35 (d, 2H, J=8.2 Hz), 7.07 (d, 2H, J=8.5 Hz), 7.04 (d, 1H, J=2.6 Hz), 6.86 (dd, 1H, J=9.1, 2.6 Hz), 6.82 (d, 2H, J=8.5 Hz), 4.24 (t, 2H, J=5.8 Hz), 3.98 (s, 2H), 3.84 (s, 3H), 3.76 (t, 2H, J=5.8 Hz). MS (ESI) m/z 500 (M+H)⁺.

### D. 3-(4-chlorophenyl)-4-(4-(2-bromoethoxy)benzyl)-7-hydroxychromen-2-one

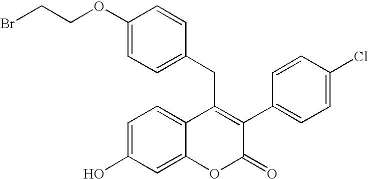

A solution of 3-(4-chlorophenyl)-4-(4-(2-bromoethoxy)benzyl)-7-methoxychromen-2-one (16.5 grams, 33 mmol) and 150 mL of 30% HBr/HOAc in a sealed tube was warmed at 100° C. for 8 h. The reaction mixture was cooled to room temperature and poured into 300 mL of water. The resulting solid was collected by filtration and purified using flash chromatography to provide the desired product (12.5 g, 78%). ¹H NMR (300 MHz, DMSO) 10.55 (s, 1H), 7.47 (d, 2H, J=8.5 Hz), 7.43 (d, 1H, J=8.8 Hz), 7.33 (d, 2H, J=8.5 Hz), 7.05 (d, 2H, J=8.5 Hz), 6.83 (d, 2H, J=8.5 Hz), 6.75 (d, 1H, J=2.2 Hz), 6.70 (dd, 1H, J=8.8, 2.2 Hz), 4.24 (t, 2H, J=5.7 Hz), 3.94 (s, 2H), 3.76 (t, 2H, J=5.7 Hz). MS (ESI) m/z 486 (M+H)⁺.

### E. 3-(4-chlorophenyl)-4-(4-(2-pyrrolidin-1-ylethoxy)benzyl)-7-hydroxychromen-2-one hydrochloride

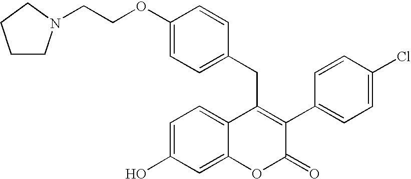

A solution of 3-(4-chlorophenyl)-4-(4-(2-bromoethoxy)benzyl)-7-hydroxychromen-2-one (8.3 g, 17.2 mmol) in 200 mL of THF was treated with 8 mL of pyrrolidine and the reaction mixture was heated at reflux for 5 h. The reaction mixture was concentrated and the crude product was purified using flash chromatography. The product was suspended in 250 mL of acetone and 4 mL of 5 M HCl(aq) was added. The mixture was stirred at room temperature overnight and the resulting solid was collected by filtration. The solid was suspended in 200 mL of ethyl acetate and the suspension was heated at reflux for 2h. The solution was cooled to room temperature and the final product was collected by filtration and dried under vacuum. The final yield was 4.96 grams (56%). ¹H NMR (300 MHz, DMSO) 10.62 (s, 1H), 10.42 (s, 1H), 7.47 (d, 2H, J=8.5 Hz), 7.43 (d, 1H, J=8.8 Hz), 7.34 (d, 2H, J=8.5 Hz), 7.09 (d, 2H, J=8.5 Hz), 6.87 (d, 2H, J=8.5 Hz), 6.77 (d, 1H, J=2.5 Hz), 6.71 (dd, 1H, J=2.5, 8.8 Hz), 4.26 (t, 2H, 4.9 Hz), 3.96 (s, 2H), 3.59-3.51 (m, 4H), 3.15-3.02 (m, 2H), 2.03-1.88 (m, 4H). MS (ESI) m/z 476 (M+H)⁺.

### Example 6

### 3-(2,4-dichlorophenyl)-4-(4-(2-pyrrolidin-1-yl-ethoxy)benzyl)-7-hydroxychromen-2-one

### A. 1-(2-hydroxy-4-methoxyphenyl)-2-(4-hydroxyphenyl)ethan-1-one

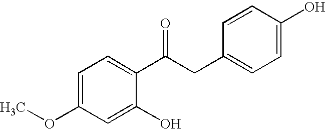

This compound was prepared using the methodology described above in Example 4A.

### B. 3-(2,4-dichlorophenyl)-4-(4-hydroxybenzyl)-7-methoxychromen-2-one

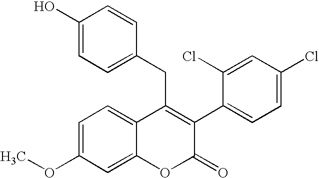

This compound was prepared using the methodology described above in Example 4B. 20 grams ketone (77.5 mmol) and 31.6 grams acid (155 mmol) provided 27.52 grams product (83%). ¹H NMR (300 MHz, DMSO-d6) 9.26 (s, 1H), 7.58 (d, 1H, J=8.8 Hz), 7.50 (dd, 1H, J=1.9, 8.2 Hz), 7.45 (d, 1H, J=8.2 Hz), 7.06 (d, 1H, J=2.2 Hz), 6.90 (d, 3H, J=8.2 Hz), 6.59 (d, 2H, J=8.2 Hz), 3.98 (d, 1H, J=15.4 Hz), 3.85 (s, 3H), 3.69 (d, 1H, J=15.4 Hz). MS (ESI) m/z 428 (M+H)⁺.

### C. 3-(2,4-dichlorophenyl)-4-(4-(2-pyrrolidin-1-ylethoxy)benzyl)-7-methoxychromen-2-one

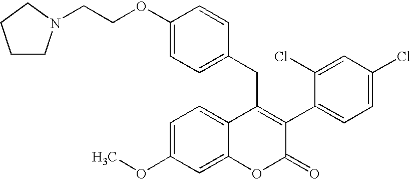

This compound was prepared using the methodology described above in Example 4C. (27.5 grams (64 mmol) of 3-(2,4-dichlorophenyl)-4-(4-hydroxybenzyl)-7-methoxychromen-2-one provided 13.5 grams product, 40% yield). ¹H NMR (300 MHz, DMSO-d6) 7.75 (d, 1H, J=1.8 Hz), 7.57 (d, 1H, J=8.9 Hz), 7.51 (dd, 1H, J=1.8, 8.2 Hz), 7.47 (d, 1H, J=8.2 Hz), 7.07 (d, 1H, 2.5 Hz), 7.02 (d, 2H, J=8.7 Hz), 6.89 (dd, 1H, J=2.5, 8.9 Hz), 6.78 (d, 2H, J=8.7 Hz), 4.04 (d, 1H, J=15.4 Hz), 3.98 (t, 2H, J=5.8 Hz), 3.85 (s, 3H), 3.74 (d, 1H, J=15.4 Hz), 2.79 (t, 2H, J=5.8 Hz), 2.57-2.52 (m, 4H), 1.69-1.65 (m, 4H). MS (ESI) m/z 525 (M+H)+.

### D. 3-(2,4-dichlorophenyl)-4-(4-(2-pyrrolidin-1-yl-ethoxy)benzyl)-7-hydroxychromen-2-one

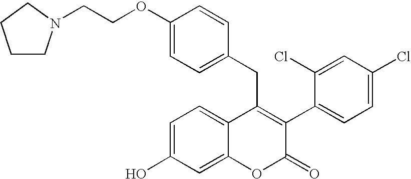

This compound was prepared using the methodology described above in Example 4D. (4.5 grams of 3-(2,4-dichlorophenyl)-4-(4-(2-pyrrolidin-1-ylethoxy)benzyl)-7-methoxychromen-2-one (8.5 mmol) provided 3.2 grams product, 73% yield).

¹H NMR (300 MHz, CDCl₃) 7.49 (d, 1H, J=1.9 Hz), 7.35 (d, 1H, J=8.5 Hz), 7.27 (s, 1H) 7.23 (dd, 1H, J=2.2, 8.2 Hz), 7.04 (d, 1H, J=8.2 Hz), 6.81 (d, 2H, J=8.5 Hz), 6.67 (s, 1H), 6.65 (dd, 1H, J=2.2, 8.5 Hz), 6.56 (d, 2H, J=8.5 Hz), 3.99 (d, 1H, J=15.6 Hz), 3.97 (t, 2H, J=5.8 Hz), 3.71 (d, 1H, J=15.6 Hz), 2.73 (t, 2H, J=6.0 Hz), 2.51-2.46 (m, 4H), 1.68-1.63 (m, 4H). MS (ESI) m/z 511 (M+H)⁺.

### Example 7

### Additional Representative Compounds

Table 1, below, discloses representative benzopyranone compounds. These benzopyranone compounds can be obtained using the methods disclosed herein.

TABLE 1
Representative Benzopyranone Compounds

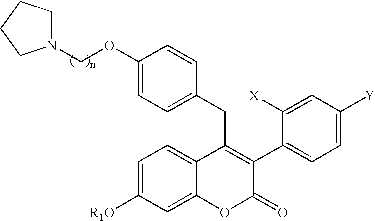

| No. | R₁ | X | Y | n |
|---|---|---|---|---|
| 1 | H | F | CF₃ | 2 |
| 2 | H | Br | CF₃ | 2 |
| 3 | H | I | CF₃ | 2 |
| 4 | C(═O)CH₃ | H | CF₃ | 2 |
| 5 | C(═O)CH₃ | Cl | CF₃ | 2 |
| 6 | C(═O)CH₃ | F | CF₃ | 2 |
| 7 | C(═O)CH₃ | Br | CF₃ | 2 |
| 8 | C(═O)CH₃ | I | CF₃ | 2 |
| 9 | C(═O)CH₃ | CF₃ | CF₃ | 2 |
| 10 | H | F | Cl | 2 |
| 11 | H | Br | Cl | 2 |
| 12 | H | I | Cl | 2 |
| 13 | C(═O)CH₃ | H | Cl | 2 |
| 14 | C(═O)CH₃ | Cl | Cl | 2 |
| 15 | C(═O)CH₃ | F | Cl | 2 |
| 16 | C(═O)CH₃ | Br | Cl | 2 |
| 17 | C(═O)CH₃ | I | Cl | 2 |
| 18 | C(═O)CH₃ | CF₃ | Cl | 2 |

### Example 8

### Inhibition of IL-6 Release

Illustrative benzopyranone compounds were tested for their ability to inhibit IL-6 release from human U-2 OS osteosarcoma cells stably transfected with human ER-α. (Stein, B.; Yang, M. X. Mol. Cell. Biol 15: 4971-4979, 1995; Poli, V. et. al., EMBO J. 13:1189-1196, 1994). As a control, IL-6 release was determined from the parental non-transfected U-2 OS cell line, which does not express detectable levels of ER-α. Benzopyranone compounds having an IC₅₀<100 nM are particularly useful as bone resorption inhibitors in vivo. Accordingly, the compounds of this assay, illustrative benzopyranone compounds, are particularly useful for the treatment of osteoporosis, Paget's disease and metastatic bone cancer. These compounds are also useful as anti-cancer agents as elevated IL-6 levels are responsible for certain cancers such as multiple myeloma, prostate cancer, ovarian cancer, renal carcinoma and cervical carcinoma.

Human U-2 OS osteosarcoma cells (ATCC) were stably transfected with expression vectors for human full-length ER-α using standard molecular biology techniques. Stable subclones were generated that expressed high levels of ER-α mRNA. The expression of ER-α was confirmed using RNase protection analysis. The parental U-2 OS cells did not express any measurable amounts of ER-α.

Cells were plated into 96-well plates at a density of 80,000 cells per well in phenol red-free media with charcoal-stripped fetal calf serum. Twenty four hours later, cells were either treated with vehicle (0.2% DMSO) or test compound (0.01 B 100 nm in 0.2% DMSO). Thirty minutes later cells were stimulated with 2.5 ng/ml TNFα and 1 ng/ml IL-1. Twenty-four hours later the media supernatant was analyzed for cytokine production (IL-6) using commercially available ELISA kits following the manufacturer=s instructions. Cytokine production in the presence of vehicle (0.2% DMSO) was set to 100%. The results are expressed as IC50 (nM) values (Table 2) which is the concentration of the benzopyranone compound necessary to inhibit the production of IL-6 50% relative to the amount of IL-6 produced in the presence of vehicle. The results show that all of the illustrative benzopyranone compounds assayed show activity and, accordingly, are useful for treating or preventing bone-resorbing diseases such as osteoporosis, Paget's disease and metastatic bone cancer, and cancers such as multiple myeloma, prostate and ovarian cancer.

### Example 9

### Inhibition of MCF-7 Breast Cancer Cell Proliferation

This example shows the ability of illustrative benzopyranone compounds to inhibit 17β-estradiol-dependent growth of MCF-7 breast cancer cells in vitro and compares their activity to that of reference SERMs. MCF-7 cells represent an excellent in vitro system to study the effects of compounds on estrogen-dependent breast cancer growth. (May, F.E.B.; Westley, B. R. J. Biol. Chem. 262:15894-15899, 1987). Benzopyranone compounds having an IC₅₀<100 nM are particularly useful as anti-breast cancer agents in vivo.

MCF-7 breast carcinoma cells were plated in 24-well dishes at a density of 5×10³ cells/well in phenol-red free DMEM:F-12 (1:1) medium containing 1% antibiotics, 0.05% mercaptoethanol, 0.01% ethanolamine, 0.42 ng/mL sodium selenite and 5% charcoal-stripped FCS.

Illustrative benzopyranone compounds (0.1-1000 nM in 0.2% DMSO) and 0.1 nM 17β-estradiol were added to the cultured MCF-7 breast cancer cells for 72 h. Subsequently, 3H-labeled thymidine was added and its incorporation into cells was measured following 4h incubation. The results are expressed as IC₅₀ (nM) values (Table 2) which is the concentration of the benzopyranone compound necessary to inhibit the growth of MCF-7 breast cancer cells by 50% relative to controls. The results show that all the illustrative benzopyranone compounds assayed show activity and, accordingly, are useful for treating or preventing breast cancer in a patient.

### Example 10

### Inhibition of BG-1 Ovarian Carcinoma Cell Proliferation

This assay shows the ability of illustrative benzopyranone compounds to inhibit 17β-estradiol-dependent growth of BG-1 ovarian carcinoma cells in vitro and compares their ability to that of reference SERMs. BG-1 cells serve as a useful in vitro model for the evaluation of the effects of antiestrogenic compounds on ovarian tumor growth (Greenberger, L. M. et. al., Clin. Cancer Res. 7:3166-3177, 2001). Benzopyranone compounds having an IC₅₀<100 nM are particularly useful as anti-ovarian cancer agents in vivo.

BG-1 ovarian carcinoma cells were plated in 24-well dishes at a density of 5×10³ cells/well in phenol-red free DMEM:F-12 (1:1) medium containing 1% antibiotics, 0.05% mercaptoethanol, 0.01% ethanolamine, 0.42 ng/mL sodium selenite and 5% charcoal-stripped FCS. Illustrative benzopyranone compounds (0.1-1000 nM in 0.2% DMSO) and 0.1 nM 17β-estradiol were added to the cultured BG-1 ovarian carcinoma cells and incubated for 72 h. Subsequently, ³H-labeled thymidine was added and its incorporation into cells was measured following 4h incubation. The results are expressed as IC₅₀ (nM) values (Table 2) which is the concentration of the benzopyranone compound necessary to inhibit the growth of BG-1 ovarian carcinoma cells by 50% relative to controls. The results show that all the illustrative benzopyranone compounds assayed show activity and, accordingly, are useful for treating or preventing ovarian cancer in a patient.

TABLE 2
In vitro data

| | IC₅₀(nM) | | |
|---|---|---|---|
| Structure | IL-6(ER-α) | MCF-7 | BG-1 |
| 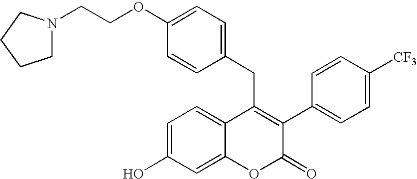 | 1.5 | 13.6 | 13.6 |
| 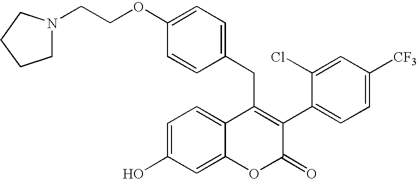 | 0.5 | 4.5 | 4.65 |
| 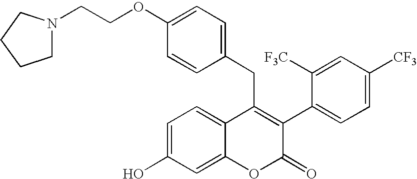 | 1.0 | 26 | 5.5 |
| 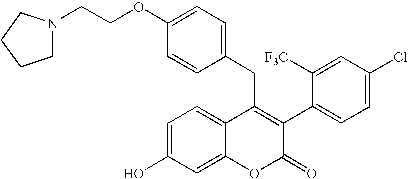 | 0.40 | 3.0 | 2.3 |
| 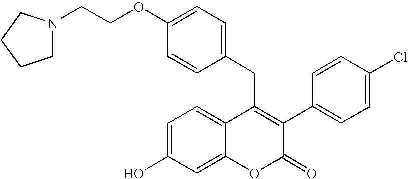 | 0.40 | 26.0 | 5.8 |
| 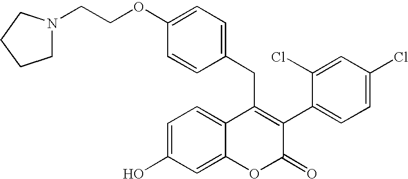 | 0.29 | 6.5 | 1.4 |

Accordingly, the in vitro results of Examples 8-10 as illustrated in Table 2 above, show that the benzopyranone compounds of the present invention are useful for the treatment or prevention of bone-resorbing diseases and various cancers.

### Example 11

### Rat Pharmacokinetic (PK) Analysis

Rat PK Cassette Standard Assay

An illustrative compound of formula (I), (II) or a pharmaceutically acceptable salt thereof, and an internal standard raloxifene is administered by oral gavage at a dose level of 5 mg/kg body weight. Blood is sampled over the time period from 15 min to 24 h postdose. Blood samples are prepared by acetonitrile precipitation, centrifuged, and supernatants are evaporated in a vacuum centrifuge. Dried residuals are dissolved in methanol/water (60:40 v/v) containing 1% formic acid and analyzed by HPLC on an UPTISPHERE™ C18 reversed-phase HPLC column (particle size: 3 μm; column dimensions: 2H 50 mm). Eluent A is 10% acetonitrile in water with 0.1% formic acid (pH 2.1), eluent B is 90% acetonitrile with 10% water and 0.1% formic acid (pH 2.1). A linear gradient is run from 5 to 100% B over 7 min followed by a 3 min hold at 100% B at a constant temperature of 50° C. in the column compartment. The flow rate is held constant at 0.4 mL/min. Sample injection volume is 10 μL. The flow from the HPLC system is directly introduced into the ion source of an Agilent 1100 series MS-detector (single quadrupole mass analyzer) and subjected to atmospheric pressure electrospray ionization (positive mode). All compounds are detected as protonated quasi-molecular ions [M+H]⁺. A structurally closely related SERM is used as an analytical internal standard. Quantification of blood levels of the compounds is based on a 7-level calibration curve (in triplicate) using blank rat blood samples to which have been added stock solutions of external and internal standards.

Rat PK Cassette Validation

Raloxifene alone is administered p.o. (3 mg/kg) to four female rats each. Blood samples are taken and analyzed as described above. The pharmacokinetic data generated from this validation study is compared with the data for raloxifene obtained in cassette dosing experiments to check for potential pharmacokinetic interactions. Deviations exceeding the typical range of biological variability (approx. +50% max. for individual parameters) are considered strongly indicative for pharmacokinetic interactions between compounds in the cassette, and the respective data are discarded.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited, the entire disclosures of which are incorporated herein by reference in their entirety.

What is claimed is:

1. A hydrochloride salt of a compound having the structure:

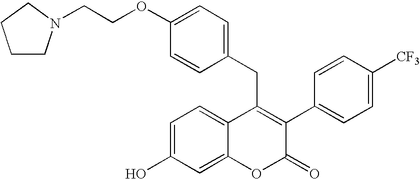

2. A pharmaceutical composition comprising a hydrochloride salt of a compound having the structure: 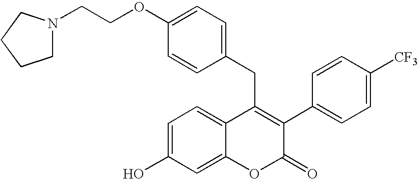
and a pharmaceutically acceptable carrier or vehicle.

3. A method for modulating gene expression in a cell expressing ER, comprising contacting the cell with an effective amount of a hydrochloride salt of a compound having the structure:

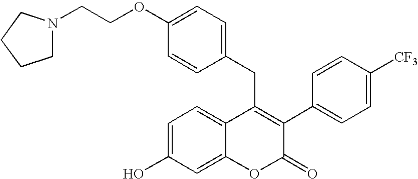

4. The method of claim 3 wherein ER is ER-α or ER-β.

5. The method of claim 3 wherein the cell preferentially expresses ER-β over ER-α.

6. The method of claim 3 wherein the cell is of bone, bladder, uterus, ovary, prostate, testis, epididymis, gastrointestinal tract, kidney, breast, eye, heart, vessel wall, immune system, lung, pituitary, hippocampus or hypothalamus.

7. A method of modulating ER in tissue expressing ER, comprising contacting the tissue with an effective amount of a hydrochloride salt of a compound having the structure:

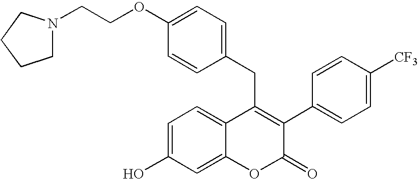

8. The method of claim 7 wherein ER is ER-α or ER-β.

9. The method of claim 7 wherein the tissue preferentially expresses ER-β over ER-α.

10. The method of claim 7 wherein the tissue is of bone, bladder, uterus, ovary, prostate, testis, epididymis, gastrointestinal tract, kidney, breast, eye, heart, vessel wall, immune system, lung, pituitary, hippocampus or hypothalamus.

11. A method for activating the function of ER in a bone cell, comprising contacting a bone cell with an effective amount of a hydrochloride salt of a compound having the structure:

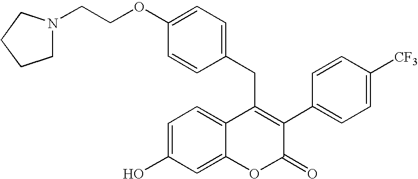

12. The method of claim 11 wherein the cell is an osteosarcoma cell.

13. A method for inhibiting the function of ER in a breast cancer cell, ovary cancer cell, endometrial cancer cell, uterine cancer cell, prostate cancer cell or hypothalamus cancer cell comprising contacting said cell with an effective amount of a hydrochloride salt of a compound having the structure:

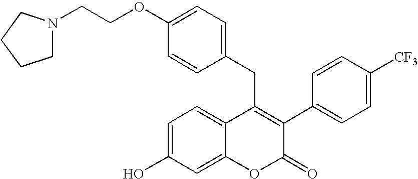

14. A method for inhibiting the expression of IL-6, comprising contacting a cell capable of expressing ER and IL-6 with an effective amount of a hydrochloride salt of a compound having the structure:

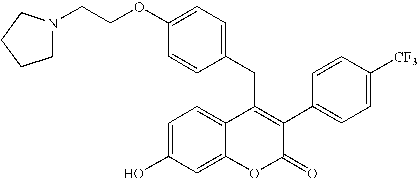

15. The method of claim 14 wherein the cell is a bone cell.

16. A method for inhibiting the growth of a cancer or neoplastic cell comprising contacting a cancer or neoplastic cell capable of expressing ER with an effective amount of a hydrochloride salt of a compound having the structure:

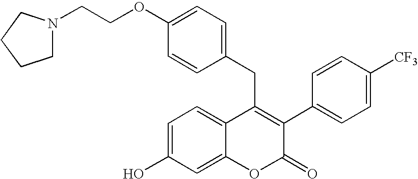

17. A method for reducing a patient's serum cholesterol level comprising administering to a patient in need thereof an effective amount of a hydrochloride salt of a compound having the structure: [chemical structure]
